(19) United States
(12) Patent Application Publication
(10) Pub. No.: US 2011/0269937 A1
(43) Pub. Date: Nov. 3, 2011

(54) Conserved-Element Vaccines and Methods for Designing Conserved-Element Vaccines

(75) Inventors: James Mullins, Seattle, WA (US); David Nickle, Seattle, WA (US); Morgane Rolland, Seattle, WA (US)

(73) Assignees: higher education, Seattle, WA (US); The University of Washington through its Center for Commercialization, a public institution of

(21) Appl. No.: 13/097,592

(22) Filed: Apr. 29, 2011

Related U.S. Application Data
(63) Continuation of application No. 11/713,474, filed on Mar. 2, 2007.

(51) Int. Cl. C07K 7/08; C07K 7/06; G06N 5/00; C07H 21/04

(52) U.S. Cl. 530/326; 530/327; 536/23.72; 530/328; 706/46

(57) ABSTRACT

Embodiments of the present invention include conserved-element vaccines and methods for designing and producing conserved-element vaccines. A conserved-element vaccine (“CEVac”) is a recombinant and/or synthetic vaccine that incorporates only highly conserved epitopes from an observed set of pathogen variants. The conserved epitopes are identified computationally by aligning biopolymer sequences, such as concatenated polypeptide sequences that together represent a pathogen proteome, corresponding to an observed set of pathogen variants, and computationally selecting conserved subsequences according to a number of subsequence-selection criteria. These subsequence-selection criteria may include a minimum conserved-subsequence length, a threshold frequency of occurrence of a particular monomer at each conserved, single-monomer position within a conserved subsequence, a threshold combined occurrence for a set of allowable variant monomers at a particular conserved, variable position within a conserved subsequence, and a maximum number of variable positions within a subsequence. A set of conserved subsequences identified according to the subsequence-selection criteria are then filtered to remove subsequences identical to, or too similar to, naturally-occurring host subsequences, and are then assembled into expression vectors for incorporation into microbial hosts for biosynthesis of a recombinant CEVac or assembled into one or more synthetic constructs for a synthetic CEVac.

### SEQUENCE PROGRAM LISTING APPENDIX

Two identical CDs identified as “Copy 1 of 2” and “Copy 2 of 2,” containing the sequence listing for the present invention, is included as a sequence listing appendix.

### TECHNICAL FIELD

The present invention is related to the design and development of recombinant, synthetic, and DNA vaccines and, in particular, to the design and development of conserved-element vaccines that prevent mutational escape, by viruses that replicate rapidly and with relatively low fidelity, from the targeted adaptive-immune response elicited by the conserved-element vaccines.

### BACKGROUND OF THE INVENTION

Recombinant, synthetic, and DNA vaccines, prepared by polypeptide or polynucleic-acid synthesis and by transforming microorganisms to produce epitope-containing polypeptides or epitope-encoding polynucleic acids, respectively, have been successfully developed for immunizing various hosts, including humans, against various pathogens, including the hepatitis-B and human papilloma viruses. Recombinant, synthetic, and DNA vaccines are particularly useful for targeting pathogens for which live or attenuated-virus vaccines are impractical or pose potential risks to vaccine recipients. Recombinant, synthetic, and DNA vaccines are also potentially more economically designed and manufactured, and can be used to address a wider range of pathogens than can be targeted by live-virus and attenuated-virus vaccines. However, the methods of the present invention may also be used in combination with virus-based or poxvirus-based delivery methods.

The human immunodeficiency virus (“HIV”), a retrovirus that causes the acquired immunodeficiency syndrome disease (“AIDS”), is one of the primary targets for current vaccine-development efforts. HIV infection in humans is now pandemic, and represents a severe and continuing health risk throughout the world. Although researchers and vaccine developers were initially hopeful of producing an effective vaccine for HIV, many years of research and development efforts have so far failed. HIV poses a number of difficult hurdles. For one thing, HIV infects the very lymphatic cells within humans that serve to help mount an immune response to destroy viral pathogens and virally infected cells. Another problem is that HIV replicates with relatively low fidelity, leading to frequent mutations and to a corresponding plethora of variant viruses within both individuals and the population as a whole. HIV can thus readily escape, by mutation, a specifically targeted immune response elicited by the prototype vaccines that have so far been prepared and tested.

Because AIDS remains a continuing and critical health threat, and because traditional vaccine design and development methods have failed to produce effective HIV vaccines, researchers and vaccine developers, public health officials, governmental agencies, health-care providers, and many health-conscious individuals have all recognized the need for new approaches to designing and developing an effective HIV vaccine. In addition, viral, bacterial, and parasitic threats continue to arise, including various strains of avian flu virus, for which vaccines may need to be developed quickly, on a massive scale, to prevent health and economic disasters. However, effective methods for controlling many well-known viruses, bacteria, and parasites have not yet been developed, despite great effort and investment. Vaccine developers, health care professionals, and the general population are acutely aware of the need for fast, economically efficient methods for developing vaccines to address fast-arising viral, bacterial, and parasitic threats.

### SUMMARY OF THE INVENTION

Embodiments of the present invention include conserved-element vaccines and methods for designing and producing conserved-element vaccines. A conserved-element vaccine (“CEVac”) is a recombinant, synthetic, and/or DNA vaccine that incorporates highly conserved sequences from an observed set of pathogen variants. In the case of a recombinant and synthetic CEVac, the conserved sequences are polypeptide sequences that are incorporated in one or more viral protein components, including viral structural and envelope proteins, proteases, transcriptases, and integrases, accessory and regulatory proteins, and other such protein and polypeptide viral components. In the case of a DNA CEVac, the sequences are nucleic-acid sequences that encode conserved protein and polypeptide viral components.

In disclosed embodiments of the present invention, the conserved sequences are identified computationally by considering biopolymer sequences, such as concatenated polypeptide sequences that together represent a pathogen proteome, corresponding to an observed set of pathogen variants, and computationally selecting, from the considered biopolymer sequences, conserved subsequences according to a number of subsequence-selection criteria. These subsequence-selection criteria may include a minimum conserved-subsequence length, a threshold frequency of occurrence of a particular monomer at each conserved, single-monomer position within a conserved subsequence, a threshold combined occurrence for a set of allowable variant monomers at a particular conserved, variable position within a conserved subsequence, and a maximum number of variable positions within a subsequence. A set of conserved subsequences identified according to the subsequence-selection criteria is then filtered to remove subsequences identical to, or too similar to, naturally-occurring host subsequences, to remove subsequences that may be immunodominant with respect to conserved subsequences more effective in eliciting an immune response, and to remove subsequences that fail, for other reasons, to effectively elicit a protective immune response or that elicit undesired responses. The filtered set of conserved subsequences is then assembled into expression vectors for incorporation into microbial hosts for biosynthesis of a recombinant or DNA CEvac, or assembled into one or more synthetic constructs for a synthetic CEVac.

### BRIEF DESCRIPTION OF THE DRAWINGS

FIGS. 1A-B illustrate the HIV viral particle and the HIV viral life cycle, respectively.

FIG. 2 provides an illustrated summary of the cytotoxic-T-cell lymphocyte-based adaptive immune response to virally infected host cells.

FIG. 3 shows the chemical structure of a small, four-subunit, single-chain oligonucleotide, or short DNA polymer.

FIG. 4 illustrates a polypeptide or protein.

FIGS. 5A-B illustrate DNA transcription and mRNA translation.

FIG. 6 illustrates the process by which a DNA mutation leads to a change in the amino-acid sequence of a polypeptide encoded by the DNA.

FIG. 7 illustrates the rapid generation of variant, mutant HIV viruses.

FIG. 8 illustrates the general theory of CEVac design.

FIG. 9 is a flow-control diagram illustrating a method for CEVac design that represents one embodiment of the present invention.

FIG. 10 illustrates the types of subsequence-selection criteria that may be applied to proteome sequences within a two-dimensional proteome-sequence array, discussed in FIG. 8, in order to identify conserved subsequences.

### DETAILED DESCRIPTION OF THE INVENTION

The present invention is directed to conserved-element vaccines and methods for designing and producing conserved-element vaccines. In the following discussion, an embodiment of the present invention directed to CEVac vaccines directed to HIV is discussed. However, it should be noted that the present invention is applicable to designing and producing recombinant, synthetic, and DNA vaccines directed to any of a large number of pathogen targets for use in any of a large number of animal and human hosts.

### HIV

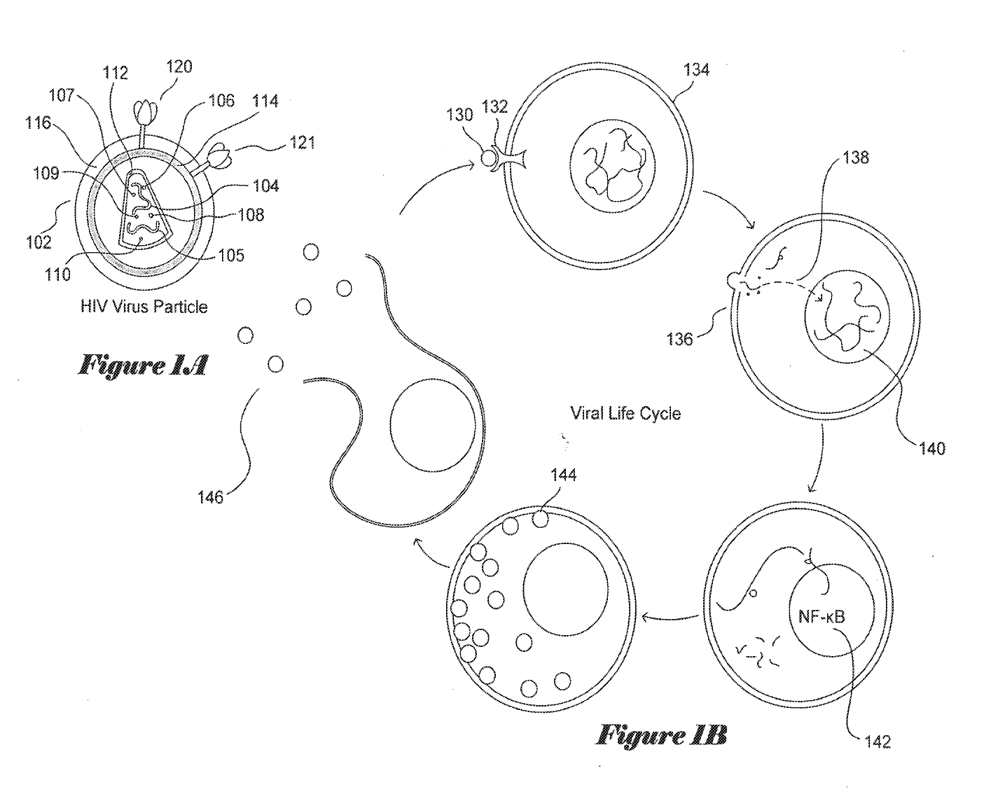

FIGS. 1A-B illustrate the HIV viral particle and the HIV viral life cycle, respectively. The HIV viral particle 102 is about 120 nanometers in diameter and is roughly spherical. The HIV viral particle includes two copies of positive, single-stranded viral RNA 104-105 that encodes the nine HIV viral genes, as well as enzymes 106-110 needed for viral integration and replication, including reverse transcriptase, a protease, and an integrase. The RNA and enzymes are enclosed by a conical capsid 112 composed of approximately 2,000 copies of the HIV protein p24. The conical capsid is, in turn, enclosed by a matrix 114 comprising the HIV protein p17 that is, in turn, surrounded by a viral envelope 116 comprising the viral surface (glycoprotein-gp120) and transmembrane (glycoprotein-gp41) proteins along with host phospholipid molecules and other host genome-encoded proteins obtained from host-cell membranes. Each viral particle includes about 70 proteinaceous protrusions, two of which 120-121 are shown in FIG. 1A. The protrusions each consist of a three-molecule-glycoprotein-gp120 cap affixed to a three-molecule-glycoprotein-gp41 anchor.

Of the nine HIV genes, two genes, gag and env, encode the structural proteins for the viral particle. The gag gene encodes structural proteins, including among others, p24, and p17. The gene env encodes a gp160 protein that is cleaved by a viral enzyme to produce the gp120 and gp41 proteins that together make up the protrusions 120 and 121. The gene pol encodes viral reverse transcriptase, integrase, and an RNase, whereas the remaining genes encode auxiliary and regulatory molecules needed to orchestrate viral replication and other functions. A gene spanning the gag-pol gene border encodes the viral protease.

HIV infects various immune-system cells, including macrophages and CD4⁺T-cells. In a first step, shown in FIG. 1B, a viral particle 130 binds to a receptor 132 on the surface of a macrophage or CD4⁺T-cell. The binding involves association of the gp120 cap and CD4 and chemokine receptors on the surface of the macrophage or CD4⁺ T-cell. Following stable association of gp120 with both a CD4 and chemokine receptor, the N-terminal portion of the gp41 viral protein penetrates the host cell membrane and mediates fusion of the viral membrane 116 and the host cell membrane 134, eventually allowing the contents of the viral particle to be released into the host cell 136. The viral reverse transcriptase enzyme copies the viral RNA into complementary DNA, and copies the initial DNA to complementary DNA to form a double-stranded viral DNA intermediate (“vDNA”) which is then transported 138 into the host-cell nuclus 140 where the vDNA is incorporated into the host cell's DNA by the viral enzyme integrase. Once incorporated into the host-cell genome, the viral DNA may remain dormant until the macrophage or T-cell is activated by a cellular transcription factor, such as the transcription factor NF-κB 142. The activated T-cell then begins to transcribe the viral-DNA-containing host genome, as a result of which the viral DNA is transcribed by the host-cell transcription machinery to produce many copies of vDNA-directed mRNA. Initially, the copied vDNA-directed mRNA is cleaved into smaller mRNA molecules that are translated by the host-cell mRNA-translation machinery to produce viral regulatory proteins from the tat and rev genes. As the rev gen product accumulates, it begins to inhibit viral mRNA cleavage, leading to translation of structural proteins gag and env from the full-length viral mRNA. As the viral structural proteins accumulate within the cell, they are assembled and transported to the plasma membrane, e.g. nascent viral particle 144 in FIG. 1B, and the completed viral particles either bud from the host-cell membrane or are released, en mass 146, upon lysis of the host cell.

### Adaptive Immune Response to Viral Pathogens

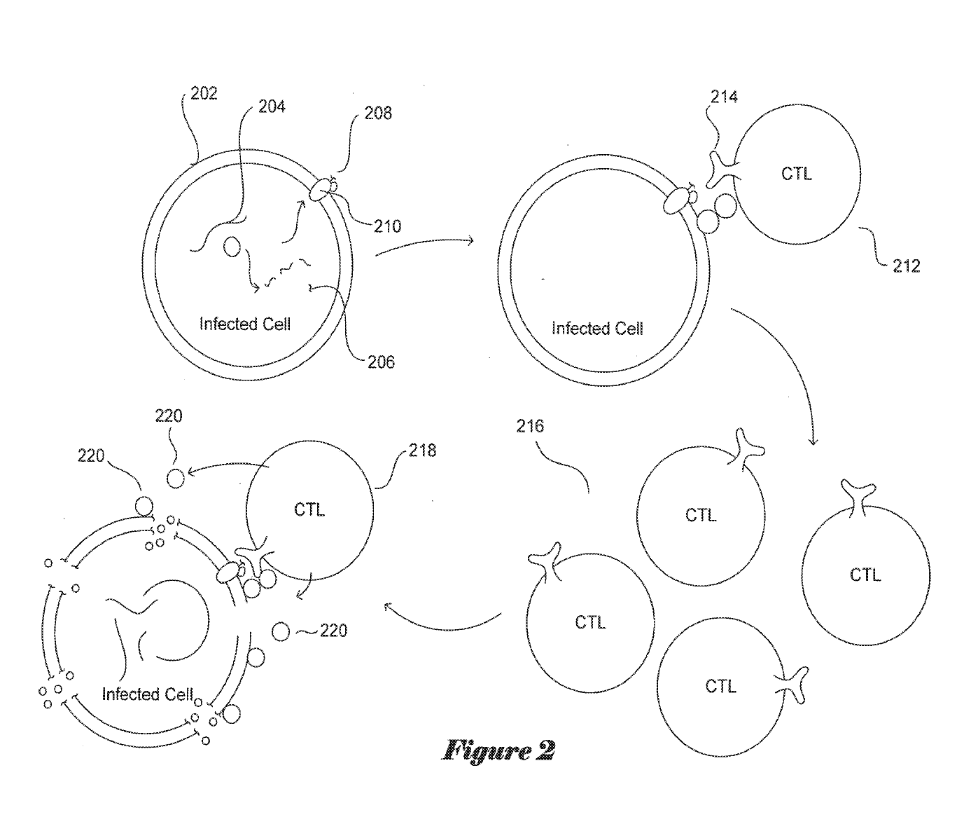

FIG. 2 provides an illustrated summary of the cytotoxic-T-cell lymphocyte-based adaptive immune response to virally infected host cells. In a virally infected host cell 202, viral as well as host-cell proteases and transport mechanisms cleave viral proteins 204 into small polypeptides, such as polypeptide 206, which are transported to the cell membrane and presented 208 on the external surface of the cell by major-histocompatibility-complex (“MHC”) Class 1 molecules 210. The human leukocyte antigen (“HLA”) system is the human MHC. An infected cell presenting viral-protein-derived polypeptides via this mechanism is referred to as an antigen-presenting cell (“APC”).

Cytotoxic T-cells (“CTL”) 212, also known as killer T-cells, represent a subgroup of the T lymphocytes, a type of white-blood cell, capable of killing virally infected host cells or transformed host cells. CTL cells are produced in the bone marrow and migrate to the thymus, where they undergo complex genetic recombination to produce a large variety of different types of CTL cells bearing specific receptors 214. CTL cells with stable antigen-specific receptors and CD8 co-receptors are selected for maturation and release by the thymus. The thymus selects CTL cells that exhibit positive binding to foreign antigens as well as weak or no binding to host-cell biopolymer subsequences, so that the mature CTL cells released by the thymus specifically target APCs presenting foreign polypeptides rather than normal host polypeptides and other host molecules. A molecule that elicits an immune response, such as a foreign protein that is cleaved into peptide fragments that are presented by APCs recognized by CTL cells, is referred to as an “epitope.”

When a mature CTL cell bearing a particular antigen-specific receptor 212 binds to a specific foreign peptide complementary to its receptor 214, and upon further, stable binding via a CD8 co-receptor, the CTL cell undergoes clonal expansion to vastly increase the number of circulating CTL cells 216 bearing the particular antigen-specific receptor. These circulating CTL cells can then migrate throughout host tissues to search for, and kill, APCs presenting the foreign antigen specifically recognized by the CTL cells. When a CTL cell recognizes an APC presenting the foreign antigen complementary to the CTL cell receptor 218, the CTL cell releases the cytotoxins perforin and granulysin 220 that cause formation of pores in the APC's plasma membrane that eventually lead to lysis of the APC. Killer-T-cell recognition of pathogen-infected host cells may be enhanced by circulating antibodies, produced by B lymphocytes, that are activated by an MHC-Class-II antigen-presentation mechanism, which bind to foreign antigens and which are, in turn, recognized by killer cells and phagocytes.

MHC-Class-II molecules present peptide fragments derived from intravesicular pathogens and extracellular pathogens to CDR-receptor containing T cells. One type of CD4-containing T cell, the T_{H}1 helper T cell, recognizes antigen bound to MHC-Class-II molecules on the surface of a macrophage, and activates the macrophage to engulf and kill bacteria that produce the antigen. T_{H}1 T cells may also release cytokines and chemokines to attract macrophages to a site of infection. Another type of CD4-containing T cell, the T_{H}2 helper T cell, recognizes antigen bound to MHC-Class-II molecules on the surface of B cells, and activates the B cell to proliferate and differentiate into antibody-producing plasma cells. Antibodies produced by antibody-producing plasma cells circulate in the blood plasma. Antibodies comprise four polypeptides that aggregate together and are linked by disulphide bonds. A portion of an antibody molecule is complementary to, and binds, a particular antigen. By binding to antigen-containing bacteria and viruses, antibodies facilitate their neutralization and/or destruction. Neutralization occurs when the antibody binds to a bacterium or virus and thereby interferes with the ability of the bacterium or virus to infect host cells. However, in general, bound antibodies elicit destruction of their targets by phagocytes, either directly, or by recruiting complement molecules to coat the target. In certain cases, recruited complement can directly kill bacteria.

The human genes for the principle MHC-Class-I and MHC-Class-II component molecules are located on chromosome 6. These genes are often referred to as the human leukocyte antigen (“HLA”) genes. Each MHC molecule comprises a number of component polypeptides, and there are multiple genes for each of these component polypeptides, each encoding different versions of the component polypeptides. As a result, in each individual, there are multiple different MHC-Class-I and MHC-Class-II molecules, each with different peptide-binding properties, and each thus capable of presenting a different range of antigen fragments. Furthermore, the MHC genes are polymorphic, with many different variants present within the human population, leading to a quite broad range of antigen-presenting characteristics within the human population. The MHC-Class-I and MHC-Class-II molecules present in a given individual may thus differ from those of another individual in antigen-fragment-presentation characteristics. As a result, a single polypeptide-based vaccine may elicit different immune responses in different individuals, due, in part, to the differences in antigen-fragment presentation by the MHC-Class-I and MHC-Class-II molecules in the different individuals. In other words, a given foreign-molecule or foreign-molecule fragment may only elicit an immune response in those individuals with particular MHC-Class-I and/or MHC-Class-II molecules that can bind to the foreign-molecule or foreign-molecule fragment. For a vaccine to be useful, is should be directed to a target bacterium or virus to effectively raise an immune response to a particular foreign target molecule across a range of individuals selected from the human population. The vaccine generally needs to contain a sufficient number of target-molecule fragments that generate peptide fragments with specific affinities to particular MHC-Class-I and/or MHC-Class-II molecule variants to ensure that an MHC-Class-I and/or MHC-Class-II molecule variant in each individual can present a peptide fragment derived from the target-molecule fragments. Alternatively, it needs to contain fewer, more broadly effective target-molecule fragments, peptide fragments of which can be presented by many different MHC-Class-I and/or MHC-Class-II molecule variants. Because of the large numbers of MHC-Class-I and/or MHC-Class-II molecule variants, more broadly effective target-molecule fragments are desirable.

Although MHC class I alleles are extremely polymorphic, with more than 800 alleles for HLA-A and HLA-B already reported in humans, at the functional level, most HLA class 1 A and B alleles can be classified into 9 different groups or supertypes. The supertypes are characterized by overlapping peptide binding motifs and repertoires. Thus, selecting peptide fragments effective with respect to the binding motifs known for all 9 supertypes can provide a CEVac with a wide coverage of the population.

Certain antigen-producing B-cells and antigen-recognizing T-cells, once activated, can persist within the host to remember specific pathogens previously recognized by the host during its lifetime, so that a strong immune response can be quickly mustered should the pathogen be again detected by the host's immune system. Vaccines elicit long-term B-cell-mediated and T-cell-mediated antigen memory within a host's immune system by introducing foreign molecules into the host that are recognized as foreign molecules by the host immune system and that elicit clonal expansion of B cells and T cells.

A number of host cells infected with a particular virus may present many hundreds or thousands of different foreign polypeptides for recognition by T-cells that, in turn, lead to foreign-polypeptide-specific immune responses. For example, many hundreds of 9-amino-acid and larger polypeptides may be cleaved from the nine HIV gene products and presented by MHC Class I molecules. However, it is observed in many cases that, of the many hundreds or thousands of different possible polypeptides presented by APC cells, generally only a small number lead to clonal expansion of antigen-specific T-cells. In other words, only a portion of the many possible presented foreign antigens obtained by proteolysis of viral proteins appear to raise a strong, specifically-targeted adaptive-immune-system response at any given time. This phenomenon is known as immunodominance.

Immunodominance may not be a problem when the constrained immune response raised by a few immunodominant epitopes is sufficient to suppress and destroy a relatively static target organism. However, in the case of a rapidly evolving target, such as HIV, the immunodominance phenomenon may lead to focusing of the immune response on a limited number of viral sequences that are relatively evolutionarily plastic, or that, in other words, can mutate to alternative, variant sequences without sufficiently impacting viral fitness to inhibit viral escape from the immune response. Thus, while the initial immune response constrained by immunodominance might be initially effective against the virus, mutation of the small number of immunodominant viral sequences to variant sequences that do not elicit an immune response leads to variant virus that can infect cells and proliferate, despite the initial immune response. Such mutable, immunodominant sequences are referred to as “decoy sequences.”

### DNA, RNA, Proteins Transcription, and Translation

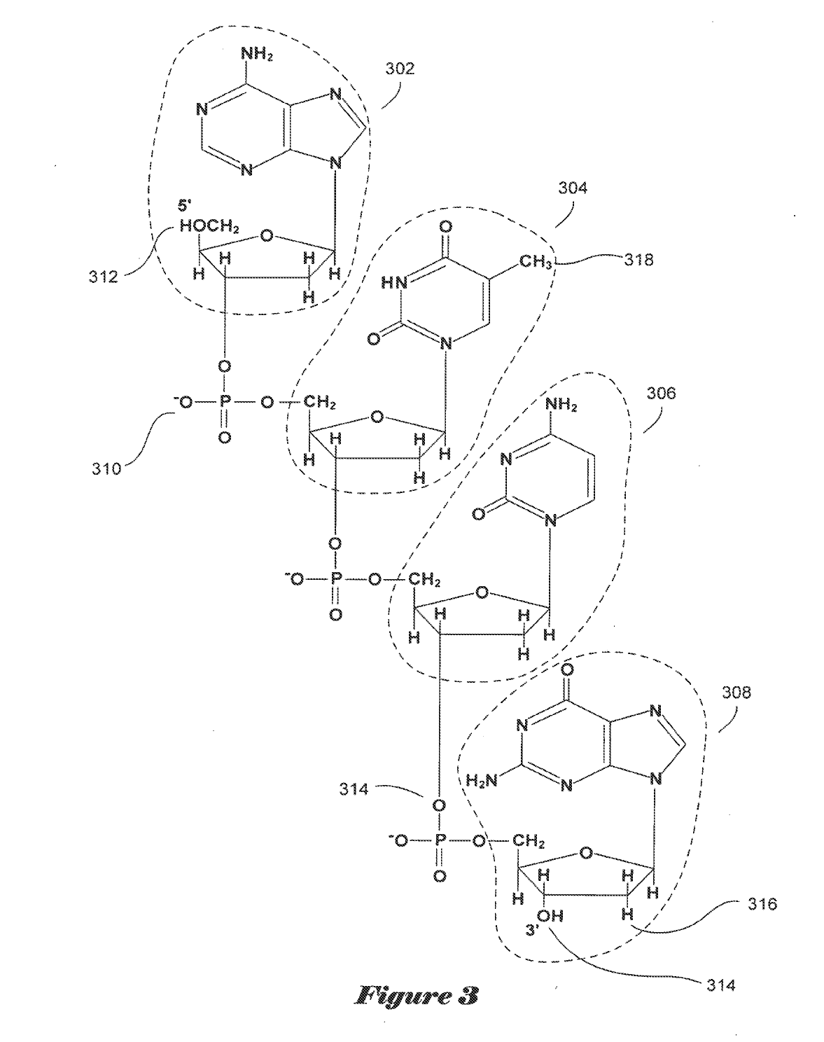

Prominent information-containing biopolymers include deoxyribonucleic acid (“DNA”), ribonucleic acid (“RNA”), including messenger RNA (“mRNA”), and proteins. FIG. 3 shows the chemical structure of a small, four-subunit, single-chain oligonucleotide, or short DNA polymer. The oligonucleotide shown in FIG. 3 includes four subunits: (1) deoxyadenosine 302, abbreviated “A”; (2) deoxythymidine 304, abbreviated “T”; (3) deoxycytidine 306, abbreviated “C”; and (4) deoxyguanosine 308, abbreviated “G.” Each subunit 302, 304, 306, and 308 is generically referred to as a “deoxyribonucleotide,” and consists of a purine, in the case of A and G, or pyrimidine, in the case of C and T, covalently linked to a deoxyribose sugar that is, in turn, linked covalently by a phosphodiester bond to a phosphate group, such as phosphate group 310. The deoxyribonucleotide subunits are linked together through phosphodiester bridges. A phosphodiester bridge is a single phosphate group through which two adjacent nucleotides are linked together via phosphoester bonds. The oligonucleotide shown in FIG. 3, and all DNA polymers, is asymmetric, having a 5′ end 112 and a 3′ end 114, each end comprising a chemically active hydroxyl group. RNA is similar in structure to DNA, with the exception that the sugar component in RNA is a ribose, having a 2′ hydroxyl instead of the 2′ hydrogen atom, such as 2′ hydrogen atom 316 in FIG. 3, and includes a ribonucleoside containing uridine instead of thymine. Uridine is similar to thymidine, but lacks the methyl group 318. The RNA subunits are abbreviated A, U, C, and G.

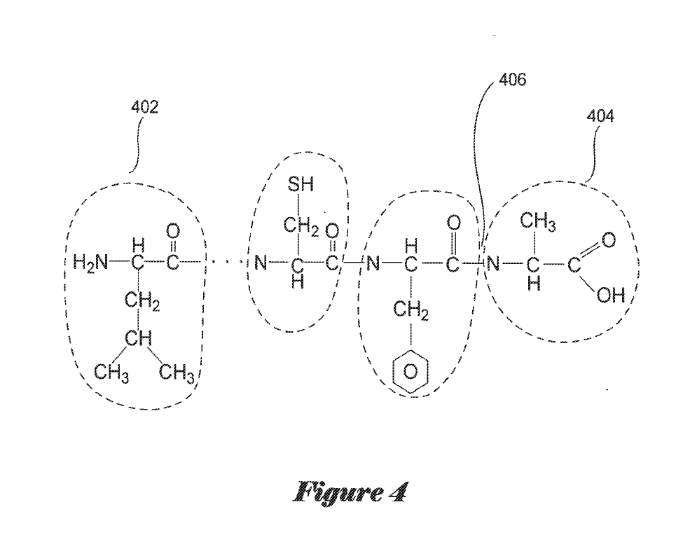

FIG. 4 illustrates a polypeptide or protein. Polypeptides and proteins are biopolymers comprising a sequence of amino-acid monomers covalently linked together by condensation reactions facilitated and directed by the ribosomal protein-synthesis machinery. A polypeptide generally has an N-terminal amino-acid monomer 402 and a C-terminal amino-acid monomer 404, with each amino-acid monomer in the polypeptide shown in FIG. 4 encircled by a dashed curve. Each internal amino-acid monomer is linked to its neighbor amino-acid monomers through an amide bond, such as amide bond 406. There are 20 common amino-acid monomers, each identified by a single-character abbreviation, such as “A” for alanine and “M” for methionine, or a three-character abbreviation, such as “ala” for alanine and “gly” for glycine. A polypeptide sequence is generally written in N-terminal to C-terminal order.

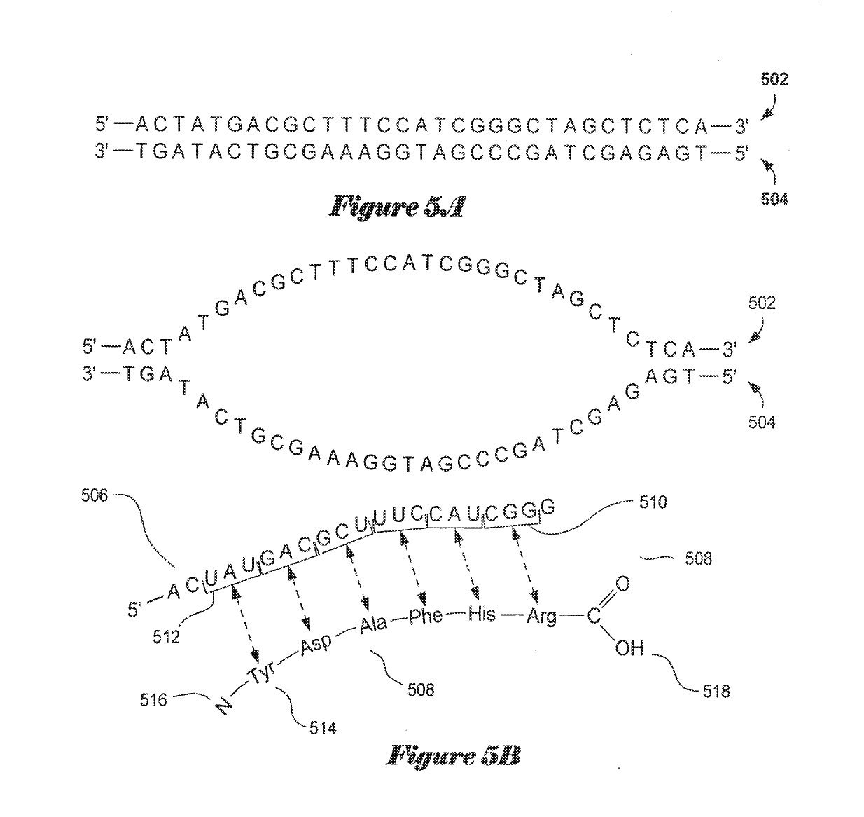

FIGS. 5A-B illustrate DNA transcription and mRNA translation. In cells, DNA is generally present in double-stranded form, in the familiar DNA-double-helix form. FIG. 5A shows a symbolic representation of a short stretch of double-stranded DNA. The first strand 502 is written as a sequence of deoxyribonucleotide abbreviations in the 5′ to 3′ direction and the complementary strand 504 is symbolically written in 3′ to 5′ direction. Each deoxyribonucleotide subunit in the first strand 502 is paired with a complementary deoxyribonucleotide subunit in the second strand 504. In general, a G in one strand is paired with a C in a complementary strand, and an A in one strand is paired with a T in a complementary strand. One strand can be thought of as a positive image, and the opposite, complementary strand can be thought of as a negative image, of the same information encoded in the sequence of deoxyribonucleotide subunits.

A gene is a subsequence of deoxyribonucleotide subunits within one strand of a double-stranded DNA polymer. One type of gene can be thought of as an encoding that specifies, or is a template for, construction of a particular protein. FIG. 5B illustrates construction of a protein based on the information encoded in a gene. In a cell, a gene is first transcribed into single-stranded mRNA. In FIG. 5B, the double-stranded DNA polymer composed of strands 502 and 504 has been locally unwound to provide access to strand 504 for transcription machinery that synthesizes a single-stranded mRNA 506 complementary to the gene-containing DNA strand. The single-stranded mRNA is subsequently translated by the cell's protein-synthesis machinery into a protein polymer 508, with each three-ribonucleotide codon, such as codon 510, of the mRNA specifying a particular amino acid subunit of the protein polymer 508. For example, in FIG. 5B, the codon “UAU” 512 specifies a tyrosine amino-acid subunit 514. The polypeptide is, as described above, asymmetrical, having an N-terminal end 516 and a carboxylic acid end 518. Other types of genes include genomic subsequences that are transcribed to various types of RNA molecules, including tRNAs, iRNAs, siRNAs, rRNAs, and other types of RNAs that serve a variety of functions in cells, but that are not translated into proteins. Furthermore, additional genomic sequences serve as promoters and regulatory sequences that control the rate, timing, and location of protein-encoding-gene expression. Although functions have not, as yet, been assigned to many genomic subsequences, there is reason to believe that many of these genomic sequences are functional. For the purpose of the current discussion, a gene can be considered to be any genomic subsequence.

In eukaryotic organisms, including humans, each cell contains a number of extremely long, DNA-double-strand polymers called chromosomes. Each chromosome can be thought of, abstractly, as a very long deoxyribonucleotide sequence. Each chromosome contains hundreds to thousands of subsequences, many subsequences corresponding to genes. The exact correspondence between a particular subsequence identified as a gene, in the case of protein-encoding genes, and the protein or RNA encoded by the gene can be somewhat complicated, for reasons outside the scope of the present invention. However, for the purposes of describing embodiments of the present invention, a chromosome may be thought of as a linear DNA sequence of contiguous deoxyribonucleotide subunits that can be viewed as a linear sequence of DNA subsequences. In certain cases, the subsequences are genes, each gene specifying a particular protein or RNA. Similarly, the HIV viral RNA, transcribed by reverse transcriptase into vDNA, represents the single genetic sequences, or genome, for the HIV virus.

### Mutation and Viral Variants

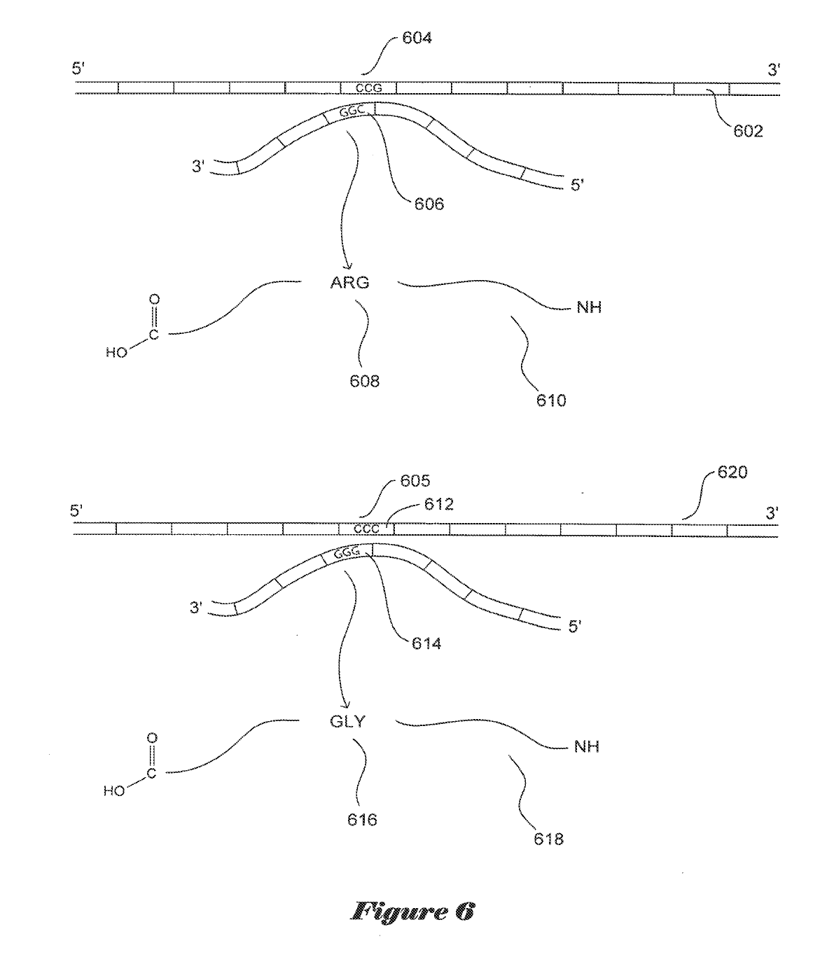

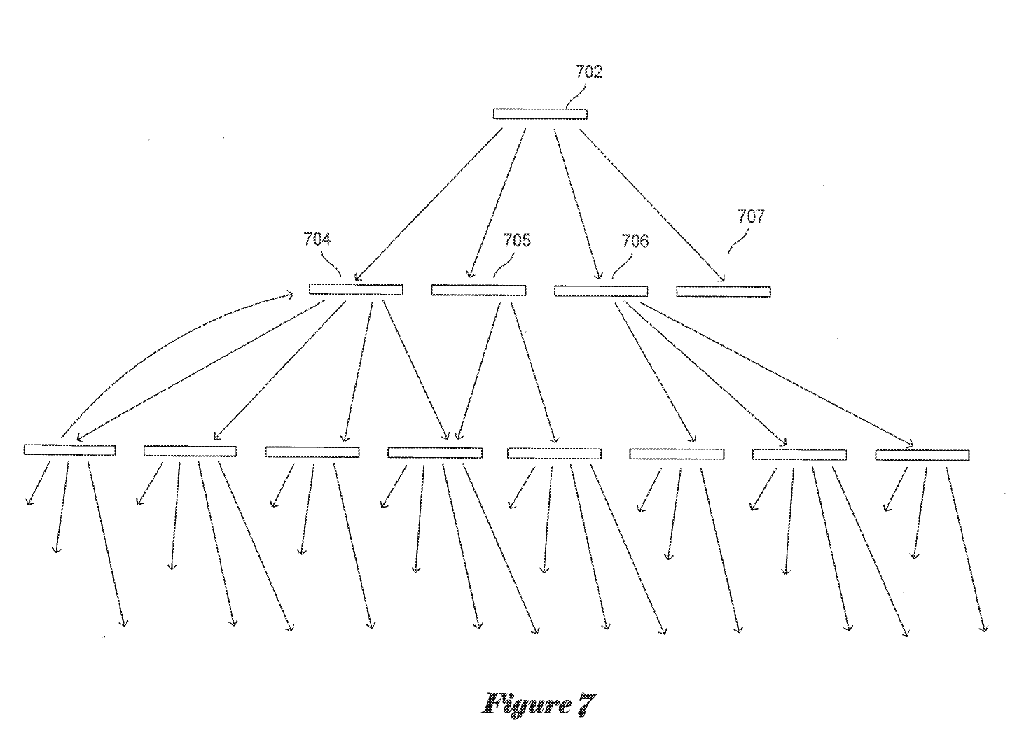

FIG. 6 illustrates the process by which a DNA mutation leads to a change in the amino-acid sequence of a polypeptide encoded by the DNA. In the top portion of FIG. 6, transcription and translation of a DNA sequence 602 is illustrated. A three-base codon 604 of the DNA sequence, CCG, is transcribed to a complementary three-base mRNA codon CGG 606 which is, in turn, translated to the amino-acid monomer arginine 608 within the polypeptide 610 corresponding to the DNA sequence 602. In the lower portion of FIG. 6, the DNA base G within the three-base codon 605 has mutated to C 612. The mutant codon is transcribed to the complementary mutant codon GGG 614, which is, in turn, translated to the amino-acid monomer glycine 616 within the mutant polypeptide 618 corresponding to the mutant DNA sequence 620. Thus, in the case shown in FIG. 6, a single nucleotide change to the original DNA sequence 602 leads to substitution of one amino-acid monomer, glycine, for the original amino-acid monomer arginine.

There are many different types of mutations. Deletion and insertion mutations may lead to frame shifts within a DNA sequence, in turn leading to changes in all or a large portion of the amino-acid monomers downstream from the amino-acid monomer corresponding to the location of the mutation. In the case of either base-substitution mutations, such as that illustrated in FIG. 6, or even in the case of multiple base-substitution mutations, the corresponding polypeptide may remain unchanged, due to redundancy in the three-base encoding of amino-acid monomers.

As briefly noted above, HIV reverse transcriptase is a relatively low-fidelity viral-RNA-to-vDNA transcription mediator. Viral reverse transcriptase has a relatively high error rate, incorporating the wrong base into the complementary DNA in about one out of every 3000 nucleotide bases transcribed. This high transcription error rate leads to frequent and diverse mutations within the vDNA. Because HIV is characterized by a relatively fast replication cycle, producing as many as 10¹⁰ or more virions per day in a human host, a single infected patient typically develops a large number of different mutation-generated HIV variant viruses, each having viral genome different from those of the other variants. FIG. 7 illustrates the rapid generation of variant, mutant HIV viruses. A single infecting viral genome 702 may suffer a number of different mutations on initial replication 704-707, each of which, in turn, may suffer additional mutations quickly leading to a large number of variant viral genomes within a very few number of replication cycles.

HIV is characterized by an enormous diversity both within hosts and at the population level, exemplified by the identification of multiple subtypes and an expanding number of circulating recombinant forms. Since HIV-1 sequences can vary by up to 30% in the envelope gene when considering only subtype B sequences, there is a considerable challenge in developing a vaccine suited for the universe of circulating strains, and there are practical limitations to the variability that can be incorporated in a vaccine.

### Viral Fitness

In general, a large number of the mutant viral genomes may correspond to less viable or completely defective viruses which cannot continue the infection cycle, and therefore represent dead ends in the evolutionary tree of mutation-produced variants. For example, mutations in sequences of structural-protein domains that interface with complementary domains in other structural proteins to form macromolecular complexes, such as viral coats and capsids, may tend to be more deleterious than mutations in sequences that do not interface with other proteins, because the architecture of binding and interface domains may be strongly constrained by that of the complementary domains, as well as by overall molecular conformation. Similarly, mutations within the sequences of the active sites of enzymes may be far more deleterious than mutations in non-catalytic domains. Mutations therefore may span a range of detrimental consequences, from innocuous, silent mutations to invariably fatal mutations. Because of the large number of infected cells and fast replication cycle, a sufficient number of viable, variant viruses with less detrimental mutations are produced by relatively low-fidelity viral transcription to generally overwhelm the host immune response. Although the host immune system may recognize and react strongly to some number of viral epitopes presented by host APCs, the high viral mutation rate generally leads to viable variants lacking the epitopes initially recognized by the immune system. Thus, HIV continues to escape host immune response directed to specific epitopes. Although many mutations may lead to virus variants that reproduce less efficiently than native virus, less fit virus variants that can nonetheless reproduce and continue the infection cycle allow the virus population to adapt to the host immune system, and avoid destruction. Additionally, less-fit viral variants may, through further mutation, revert to native virus when the immune response subsequently weakens, or may continue to evolve to produce increasingly fit variants.

Very limited sequence variation can be tolerated in some structurally and functionally important regions, like the capsid protein of HIV-1. Mutations are rare in this region. The mutations are likely to incur a substantial cost to fitness, corresponding to epitopes in which immune escape will be both very unlikely to be sustained in a host, and are likely to revert after transmission to a host without that particular restricting allele. These mutations sometimes appear in conjunction with flanking mutations that are compensatory in function, restoring fitness or preventing the proper cleavage and presentation on MHC.

### CEVac

Because of the HLA-restriction, HLA-polymorphism, and immunodominance phenomena, discussed above, specific vaccines directed to HIV generally elicit only a relatively small number of strong, epitope-directed immune responses within a given host. This allows HIV to eventually escape the immune response by producing variant viruses lacking the small number of epitopes to which the immune response is directed. Although the immune response may recognize new epitopes of variant viruses, and may continue to respond to viral mutation, the immune response lags viral escape through mutation, contributing, in most individuals, to the eventual overwhelming the individual's immune system.

Conservative-element vaccines (“CEvacs”) and methods for designing and producing CEvacs, both embodiments of the present invention, may theoretically block HIV escape of the immune-system response. In general, certain portions of a viral genome, or of any genome, are more stable towards mutation than others. For example, subsequences of critical portions of structural proteins recognized by other structural proteins in order to coalesce to form a viral capsid or protrusion or that bind to host-cell receptors, may be far more critical to viral reproduction and infectivity than polypeptide domains that do not interact with other polypeptide domains or host molecules. Mutations to these critical regions most often result in defective and non-viable viral particles. Using viral gene sequence data, segments of the viral proteins that do not, or only rarely, mutate can be identified. These segments represent candidates for immutable viral function, i.e. candidate segments for epitopic recognition that is more likely to play a protective role in HIV infection. Were it possible to develop a vaccine capable of raising a strong immune response to all, or a very large proportion of, these critical regions, it is possible that viral-mutation-directed escape of the immune-system response may be entirely prevented. In the face of a strong immune response directed to all, or a large portion of, the critical-region epitopes, a virus would need a relatively large number of simultaneous mutations in order to escape the immune response. However, as the number of mutations needed to escape the immune response increases, the likelihood of a virus incorporating the needed mutations and remaining viable exponentially decreases. Mutation-directed immune-response escape can be thought of as a path search within a huge forest of possible sequence mutations, a successful path representing only a tiny fraction of the possible mutational pathways, the overwhelming majority of which lead to defective-virus dead ends. When a virus can search the sequence space one mutation-at-a-time, the virus, because of the huge number of parallel searches made possible by the large number of infected host cells, can efficiently search the sequence space for a path of non-defective mutations leading to a sequence that escapes the immune response. However, if multiple simultaneous mutations are needed, the sequence-space search becomes intractable, because of the enormous number of possible multiple-mutation defective sequences separating a viable sequence from a next viable sequence. Thus, CEVacs may represent the best possible approach to eliciting effective immune-system control of rapidly mutating viruses, such as HIV, and may, also represent the best approach to quickly and economically subduing any of a multitude of human pathogens via recombinant and synthetic vaccines.

Effective CEVac design embodies a number of principles. First, a CEVac needs to target only conserved elements identified in target organism molecules. As a corollary, segments that can easily mutate, referred to as “decoys,” should be excluded. Decoys provide escape pathways for a virus or other pathogen, allowing the pathogen to escape the immune system by altering mutable sequences to evade an immune response directed to the current decoy sequence. Moreover, sequences that can mutate to forms resulting in a less fit, but still viable, pathogen need to be eliminated, so that a pathogen cannot temporally trade fitness or optimal function for survivability, and then, subsequently, revert to a more optimal sequence after the immune response to the more optimal sequence has subsided. An effective CEVac needs to target conserved elements present within all, or as many as possible, native viral variants currently infecting the human population. The conserved elements targeted by an effective CEVac need to be sequences that, when mutated, confer extremely deleterious or fatal consequences on the mutant virus, in order to avoid inadvertently including decoy sequences in the CEVac. The conserved elements included in an effective CEVac need to elicit an immune response across the various polymorphic MHC-Class-I and MHC-Class-II molecules present in the human population. A broad response may be obtained by broadly immunogenic conserved elements, or by including a sufficient number of less broadly effective conserved elements to elicit an immune response across a range of MHC-Class-I supertypes and MHC-Class-II molecule polymorphisms present in the human population, or within large subpopulations for which specific vaccines can be developed.

Identifying conserved elements with the above-described characteristics for a CEVac is a first step. However, CEVac design also involves packaging conserved elements effectively into one or more vaccine molecules, such as polypeptides or DNA sequences, in order to prevent inadvertent generation of host-like constructs, that might lead to autoimmune reactions, prevent inadvertent generation of decoy sequences, and in order to ensure that the conserved elements lead to effective presentation of immunogenic peptide fragments to elicit specific immune responses to the conserved elements. The packaging step may involve selecting linker sequences, positioning conserved elements correctly within the vaccine molecules, and correctly with respect to one another, including different numbers of conserved-element copies, and other such considerations.

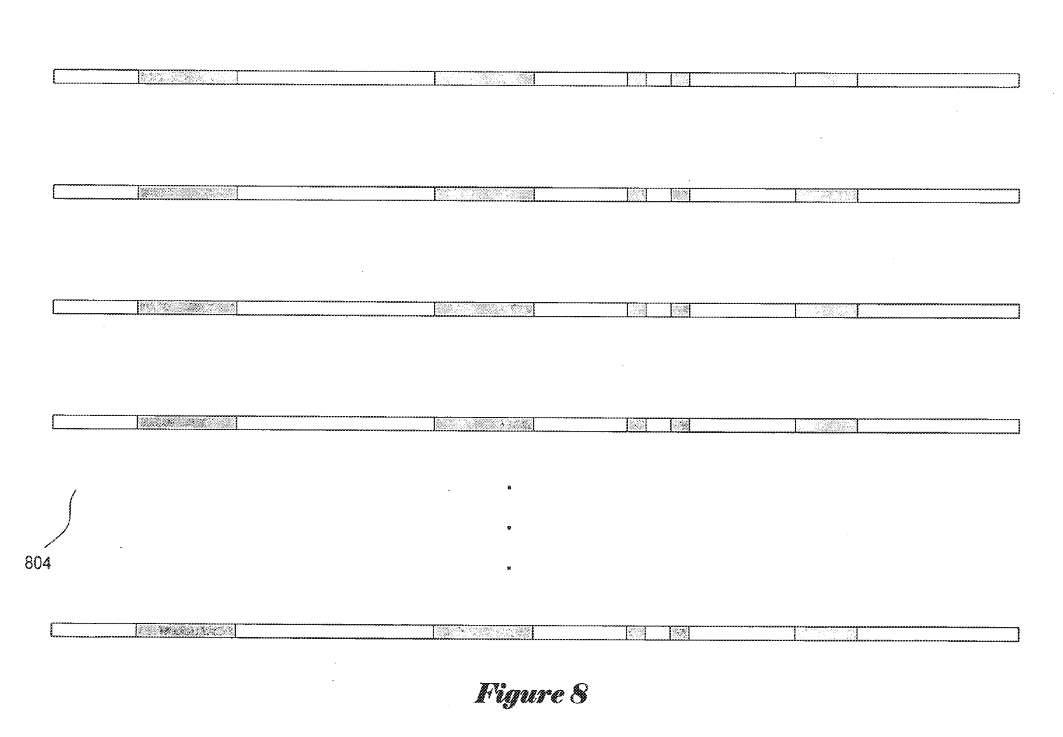

FIG. 8 illustrates the general theory of CEVac design. In FIG. 8, the polypeptide sequences for all viral proteins of a particular viral variant are coalesced together to produce a viral proteome, such as viral proteome 802, representing the total, expressed viral-variant peptide sequence. The proteomes for each identified variant virus are aligned with one another to produce a two-dimensional proteome array 804. Conserved subsequences within the proteome array are represented, in FIG. 8, by shaded portions of the proteomes, such as shaded portion 806 of proteome 802. These conserved portions of the proteome array form invariant or minimally varying subsequence columns within the two-dimensional proteome array. CEVac design involves identifying these conserved elements and then incorporating the conserved elements of the viral proteome array into a recombinant or synthetic vaccine. As discussed above, if the synthetic vaccine elicits a strong, specific immune response to all or some essential number of the conserved elements, it is likely that even a highly variable infectious agent, such as HIV, will not be able to escape immune suppression through mutation, since too many concurrent mutations would need to occur in order to escape the immune response.

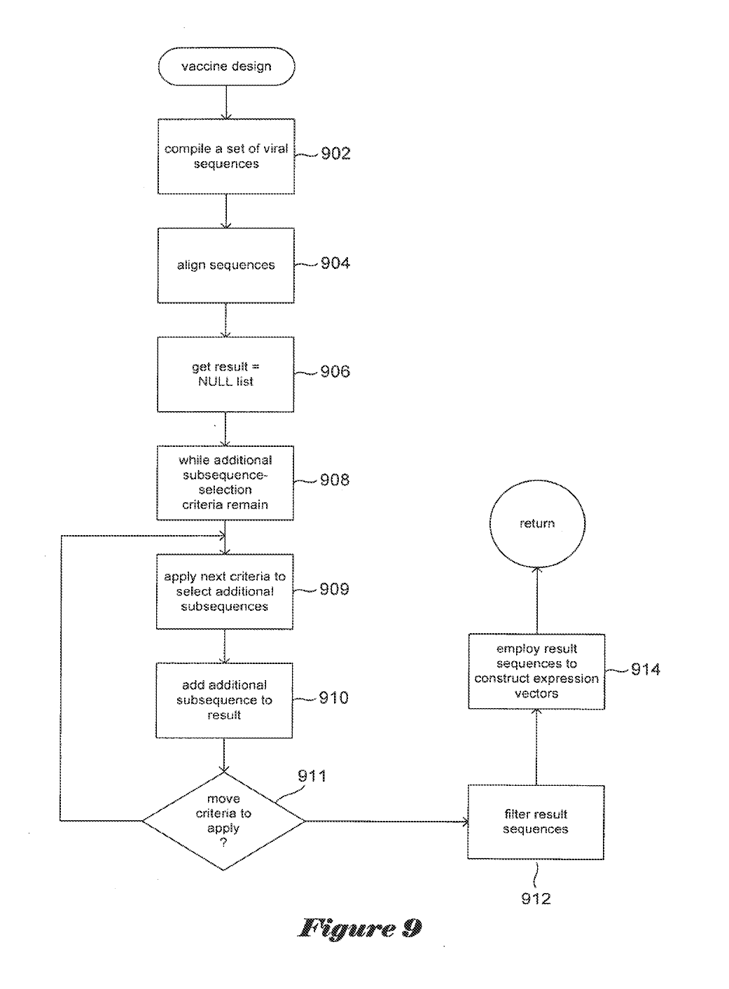

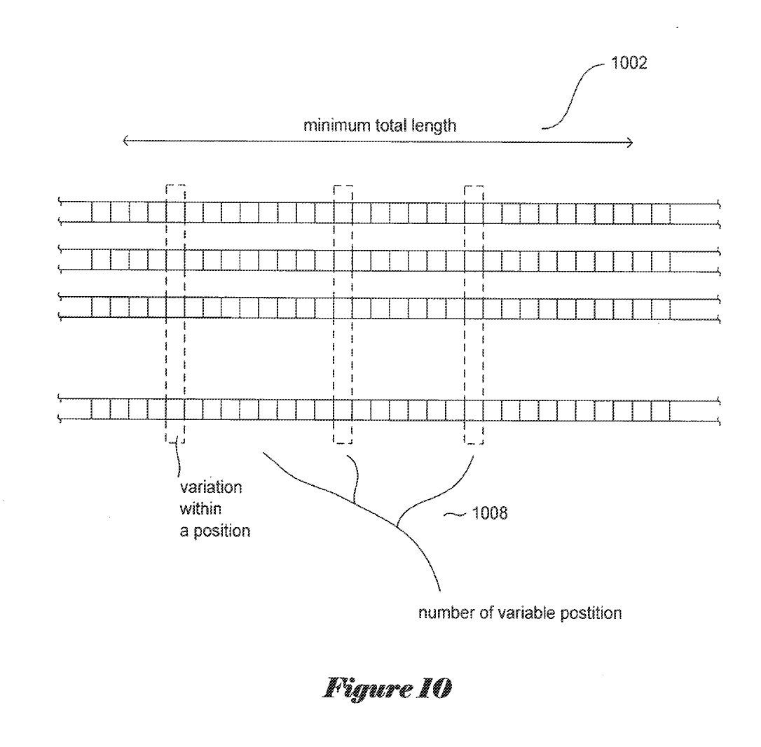

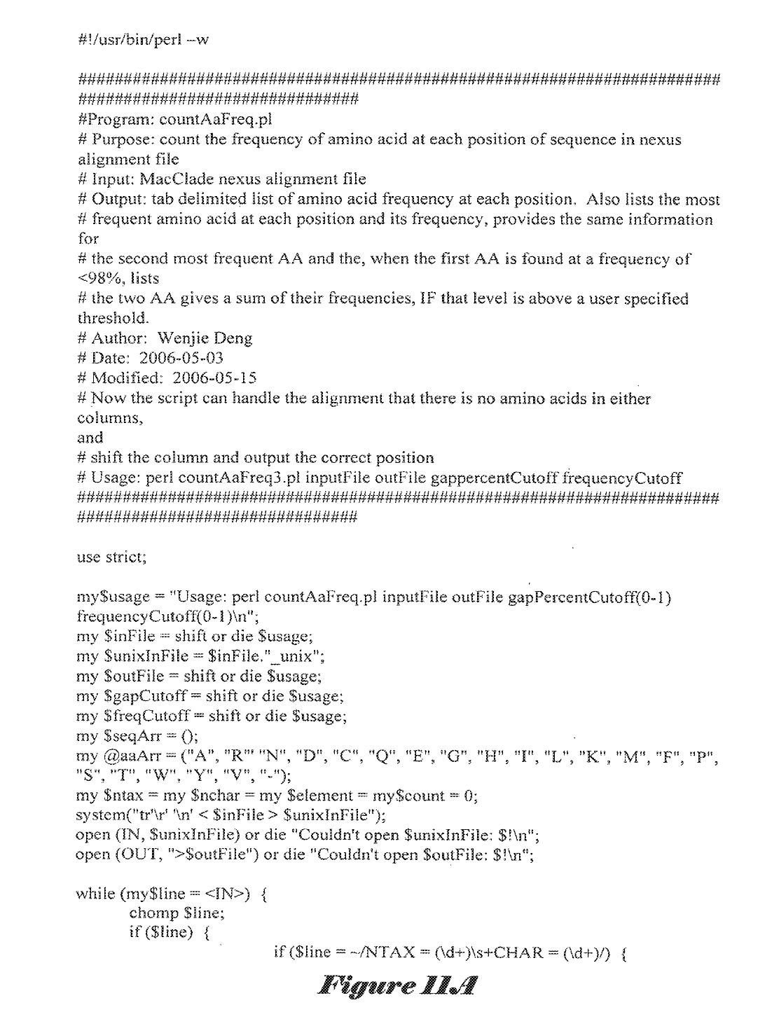

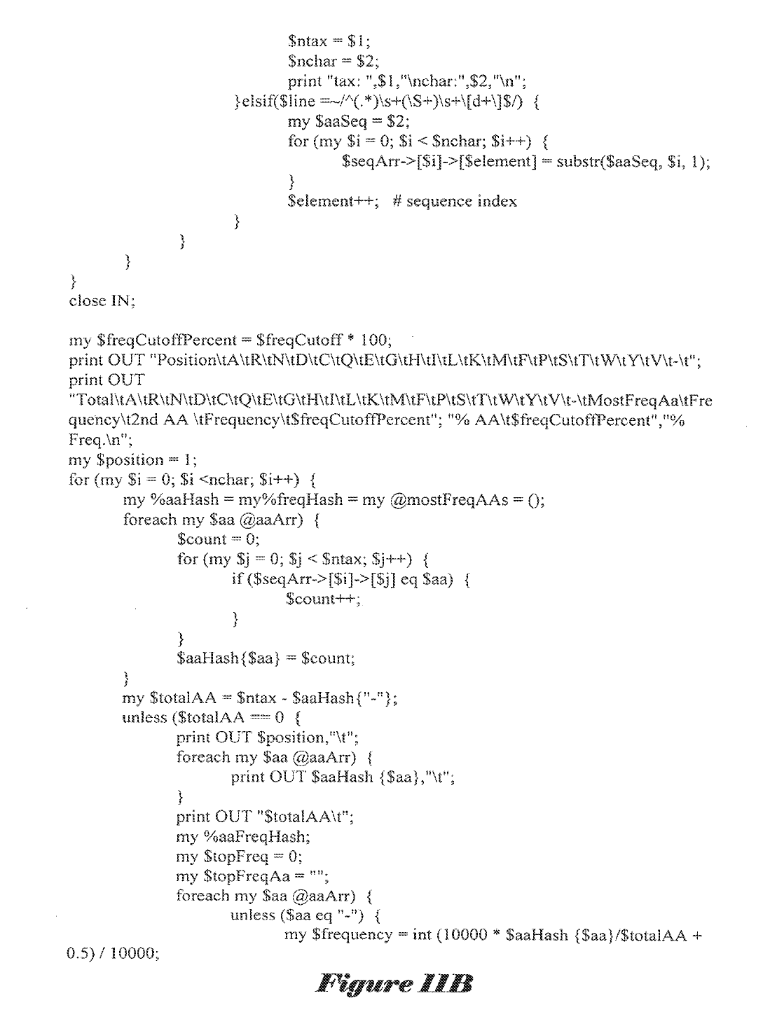

FIG. 9 is a flow-control diagram illustrating a method for CEVac design that represents one embodiment of the present invention. In a first step 902, a set of viral polypeptide sequences, or proteomes, is compiled from the sequenced proteins of all identified viral variants. Next, in step 904, the set of sequences, or viral proteomes, is aligned, by methods discussed below. Alignment places monomer positions of each of the viral proteomes in a best possible positional correspondence with one another, despite deletion, addition, and substitution mutations. Next, in step 906, a result set is set to the null set. In the while-loop of steps 908-911, each of a series of one or more subsequent-selection criteria is applied to the aligned sequences in order to identify conserved elements within the two-dimensional viral proteome array described with reference to FIG. 8 and represented by the aligned sequences produced in step 904. In step 910, any additional conserved elements identified by application of the currently considered set of subsequence-selection criteria, in step 909, are added to the result set. Next, in step 912, following termination of the while-loop, the final result set is filtered to remove sequences that may be identical to, or too similar to, naturally occurring host polypeptide sequences in the host proteome. This step is carried out in order to increase the specificity of the CEVac to viral epitopes, as well as to prevent the possibility of eliciting an autoimmune response in a vaccinated host. Finally, in step 914, the filtered sequences are employed to construct one or more expression vectors that are introduced into a microbrial host for replication and production of polypeptide sequences incorporated within a recombinant CEVac, to construct viral vectors, or to construct one or more synthetic polypeptides incorporated within a synthetic CEvac. In this final step, larger conserved subsequences may be trimmed or tailored to fit various size and sequence constraints that characterize efficient and viable polypeptide sequences for eliciting effective immune response, and conserved elements may be enhanced or modified by addition of initial and trailing subsequences for a variety of purposes.

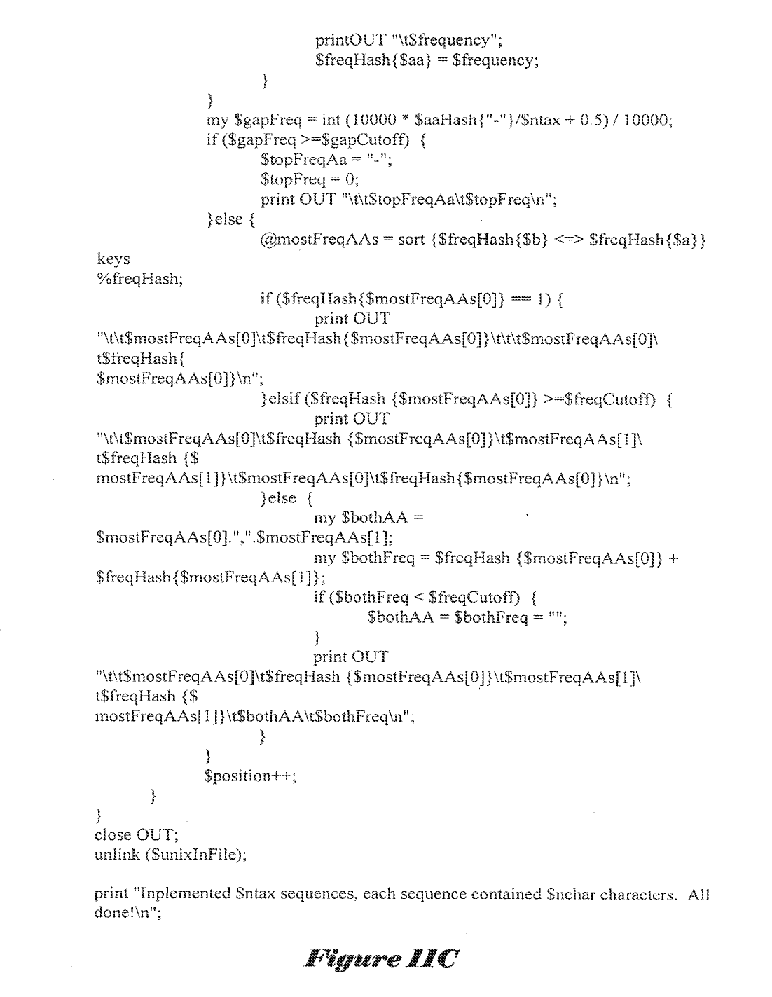

FIG. 10 illustrates the types of subsequence-selection criteria that may be applied to proteome sequences within a two-dimensional proteome-sequence array, discussed in FIG. 8, in order to identify conserved subsequences. For example, conserved subsequences may need to have a minimum total length 1002. As another example, at any given amino-acid-monomer position 1004 within the aligned proteomes, no more than a maximum amount of variation may be allowed. There may, for example, be a maximum amount of variation for a single, conserved amino acid at the position, or a maximum amount of variation for a small, selected set of amino acids that together represent a variable amino-acid monomer at that position. As another example, the number of variable amino-acid positions 1008, in contrast to positions with only a single, conserved amino acid, may need to be equal to, or less than, some maximum number of allowable variable positions. Many other types of subsequence-selection criteria may also be used. The intent of the subsequence-selection criteria is to choose maximally sized conserved regions of the proteome within which no, or minimal, amino acid variation occurs. The crux of CEVac design is to employ sufficiently restrictive criteria to identify a sufficiently small, but important set of epitopes to elicit a strong immune response to those epitopes despite the above-discussed immunodominance phenomenon.

### C++-Like Pseudocode Implementation of a CEVac Design Method

The following C++-like pseudocode provides an illustration of one embodiment of the present invention. The C++-like pseudocode is meant to illustrate one approach to implementing a conserved-element analysis program for analyzing sequences in order to find conserved elements, but is not intended to define the invention or in any way limit the scope of the claims.

First, a number of constants and an enumeration are provided:

1 const int maxPositionsPerSequence=60;

2 const int maxNumSequences=100;

3 const char NULL_CHAR=‘z’+1;

4 const int numAminoAcids=27;

5 const int maxFreqPerPos=10;

6 enum posType {conserved, variable, unconserved};

The constants “maxPositionsPerSequence and “maxNumSequences” specify the maximum number of amino-acid monomers allowed per sequence and the number of sequences that can be analyzed, respectively. The relatively small numbers used in the pseudocode are not reflective of the sizes of sequences, and numbers of sequences, that would be analyzed in an actual implementation. In the pseudocode implementation provided below, static data structures are employed, and thus relatively small sequences and numbers of sequences are used. In a more practical, robust implementation, dynamic memory allocation is employed, to provide more flexible memory usage, and the ability to dynamically allocate memory on an as-needed basis. In general, thousands of sequences may be analyzed, each of which has thousands, tens of thousands, hundreds of thousands, or millions of sequence positions. In the pseudocode embodiment, it is assumed that polypeptide sequences having amino-acid identifiers at each position are analyzed, but, in alternative embodiments, nucleic-acid sequences may be similarly analyzed, and, in yet further embodiments, various other biopolymers may be analyzed by alternative sequence-analysis routines.

The constant “NULL_CHAR” represents a null, or blank character that is inserted into sequences during alignment in order to insert one or more placeholders, or gaps, into the sequences. The constant “numAminoAcids” represents the number of different amino acids numerically identified for insertion into sequences and for other purposes. In general, there are 20 commonly occurring amino acids, but certain additional amino acids may be found in certain polypeptides found in various organisms. The constant “maxFreqPerPos” defines the size of a sequence-position/amino-acid-occurrence-frequency table, discussed below. The enumeration “posType” presents the classification of a position within a one-dimensional map representing the aligned sequences corresponding to the original sequences supplied for alignment, with the possible types of positions being “conserved,” “variable,” or “unconserved.”

Next, a declaration for a type of structure, “Amino_Acid_Frequency,” is defined. This structure contains a floating-point value indicating the frequency of occurrence of an amino acid, along with an integer value defining the particular amino acid.

| 1 typedef struct amino_acid_freq | |
|---|---|
| 2 { | |
| 3 | double freq; |
| 4 | int amino_acid; |

5 } Amino_Acid_Frequency;

Next, the class “compatibleAminoAcids” is declared:

| | | Next, the class “compatibleAminoAcids” is declared: |
|---|---|---|
| 1 class compatibleAminoAcids | | |
| 2 { | | |
| 3 | private: | |
| 4 | | bool aminoAcids[numAminoAcids]; |
| 5 | | |
| 6 | public: | |
| 7 | | void add(char aminoAcid) {aminoAcids[aminoAcid − ‘a’] = true;}; |
| 8 | | void add(char* c, int len) |
| 9 | | {for (int i = 0; i < len; i++) add(c[i]);}; |
| 10 | | void del(char aminoAcid) {aminoAcids[aminoAcid − ‘a’] = false;); |
| 11 | | bool in(char aminoAcid) {return (aminoAcids[aminoAcid − ‘a’]);}; |
| 12 | | compatibleAminoAcids( ); |
| 13 }; | | |

The instance of the class “compatibleAminoAcids” contains a number of amino-acid-identifying integers. The amino-acid identifiers included within an instance of the class “compatibleAminoAcids” represents a set of amino acids that can be substituted for one another at a variable position within a sequence. For example, it may be the case that it is a desire to restrict variable positions within conserved elements to include only related amino acids, such as substitutions of valine for isoleucine or other non-polar amino acids. This class includes function members for writing or deleting particular amino acids from the set represented by an instance of the class, as well as the function member “in,” declared above on line 11, which returns a Boolean value indicating whether a particular amino acid provided as an argument is included in the set of amino acids represented by the instance of the class “compatibleAminoAcids.”

Next, the class “positionAssignmentParameters” is provided:

```
  1 class positionAssignmentParameters
  2 {
```

| | |
|---|---|
| 3 | private: |

| | |
|---|---|
| 4 | double conservedThreshhold; |
| 5 | int numVariablePositions; |
| 6 | int numAAsAtVariablePosition; |
| 7 | double variableThreshhold; |
| 8 | int thresholdCELength; |

9

| | |
|---|---|
| 10 | public: |

| 11 | double getConservedThreshold( ) {return |
|---|---|
| | conservedThreshhold;}; |
| 12 | void setConservedThreshold(double t) |
| | {conservedThreshhold = t;}; |
| 13 | int getMaxVariablePositions( ) {return |
| | numVariablePositions;}; |
| 14 | void setMaxVariablePositions(int nv) |
| | {numVariablePositions = nv;}; |
| 15 | int getMaxAAsAtVariablePosition( ) {return |
| | numAAsAtVariablePosition;}; |
| 16 | void setMaxAAsAtVariablePosition(int vn) |

| | |
|---|---|
| 17 | {numAAsAtVariablePosition = vn;}; |

| 18 | double getVariableThreshhold( ) {return |
|---|---|
| | variableThreshhold;}; |
| 19 | void setVariableThreshhold(double vt) |
| | {variableThreshhold = vt;}; |
| 20 | int getThresholdCELength( ) {return thresholdCELength;}; |
| 21 | void setThresholdCELength(int tl) {thresholdCELength = |
| | tl;}; |

22 };

The instance of the class “positionAssignmentParameters” contains numerical parameters that specify a particular search for, or sequence-analysis for discovering, conserved elements. These parameters include: (1) “conservedThreshold,” the lowest frequency of occurrence of an amino acid at a particular position needed to consider the position conserved; (2) “numVariablePositions,” the number of variable positions allowed within a conserved element; (3) “numAAsAtVariablePosition,” the number of different amino acids that may occur in a single variable position; (4) “variableThreshold,” the minimum combined frequency of occurrences of the amino acids that occur at a variable position that allow the position to be considered to be a variable position; and (5) “thresholdCELength,” the minimum length, in amino-acid residues, of a conserved element. The class “positionAssignmentParameters” includes function members that allow these parameters to be entered into, and to be retrieved from, an instance of the class “positionAssignmentParameters.” It should be noted that many additional parameters, and types of constraints, may be defined in more fully specified conserved-element analysis programs representing alternative embodiments of the present invention. The five parameters chosen to define conserved-element searches in this pseudocode implementation are meant merely to illustrate the process and coding conventions by which such parameters may be defined and used to tailor a search for conserved elements. Next, a declaration for the class “sequence” is provided:

| 1 class sequence | |
|---|---|
| 2 { | |
| 3 | private: |

| | |
|---|---|
| 4 | char seq[maxPositionsPerSequence]; |
| 5 | int len; |

| 6 | |
|---|---|
| 7 | public: |

| | |
|---|---|
| 8 | sequence& operator = (sequence s); |
| 9 | char operator [ ] (int i) {return get(i);}; |
| 10 | char get(int i); |
| 11 | bool set(int i, char val); |
| 12 | int getLen( ) {return len;}; |
| 13 | void setLen(int l) {len = l;}; |
| 14 | bool set(char* s, int len); |
| 15 | bool insertNull(int i, int j); |
| 16 | sequence( ); |

17 };

An instance of the class “sequence” is simply a sequence of amino-acid identifiers, or an array of amino-acid identifiers. A sequence has a length and an ordered sequence of amino-acid identifiers, which may include the NULL_CHAR representing a gap, or space, in the sequence, and which can be set and retrieved using the function members declared in the declaration of the class “sequence,” above.

Next, the declaration for the class “sequences” is provided:

| 1 class sequences | |
|---|---|
| 2 { | |
| 3 | private: |

| | |
|---|---|
| 4 | sequence seqs[maxNumSequences]; |
| 5 | int num; |

| 6 | |
|---|---|
| 7 | public: |

| | |
|---|---|
| 8 | sequence& operator [ ] (int i); |
| 9 | sequence* getSeq(int i); |
| 10 | bool addSeq(char* sq, int ln); |
| 11 | bool addSeq(sequence* sq); |
| 12 | int addSeq( ); |
| 13 | char get(int s, int i); |
| 14 | bool set(int s, int i, char val); |
| 15 | int getNum( ) {return num;}; |
| 16 | bool setSeq(sequence* sq, int i); |
| 17 | void clear( ); |
| 18 | sequences( ); |

19 };

The class “sequences” is essentially an array of sequences. An instance of the class “sequences” may, for example, be used to contain all the original sequences to be analyzed for conserved elements, aligned versions of the original sequences, and the conserved elements identified in a conserved-element search. The function members declared for the class “sequences” include function members to add sequences to an instance of the class “sequences,” retrieve sequences from an instance of the class “sequences,” obtain the number of sequences in an instance of the class “sequences,” and to reinitialize the instance of the class “sequences” to the empty set. A special instance of the class “sequence” is declared as: sequence NULL_SEQ. This sequence is used as a return value in several member functions of the class “sequences” to indicate that no further sequences are available in a set of sequences.

Next, an instance of the class “aligner” is provided:

```
  1 class aligner
  2 {
```

| | |
|---|---|
| 3 | private: |

| | |
|---|---|
| 4 | sequences* origSeqs; |
| 5 | sequences* alignedSeqs; |

6

| | |
|---|---|
| 7 | int best; |
| 8 | int bestI, bestJ, bestSz; |

9

| 10 | double score(int i, int j); |
|---|---|
| 11 | void findBest( ); |
| 12 | bool insertNullsOnce(int i, int j, int nm); |
| 13 | bool insertNullsAllExcept(int i, int j, int nm); |
| 14 | void computeIRuns(int iStart, int jStart, int iEnd, int jEnd, |
| | int ref, int s); |
| 15 | void pairwiseAlign(int iStart, int jStart, int iEnd, int jEnd, |
| | int ref, int s); |

16

| | |
|---|---|
| 17 | public: |

| | |
|---|---|
| 18 | void align(sequences* orig, sequences* aligned); |
| 19 | aligner( ); |

20 };

The class “aligner” represents alignment functionality for aligning sequences prior to searching the aligned sequences for conserved elements. There are many different possible techniques and methods for aligning sequences. Many of these techniques and methods are quite sophisticated and employ a vastly more complex set of considerations than the alignment functionality provided in this pseudocode example. The techniques and methods employed for aligning sequences for a conserved-element search may significantly impact the results of the search, so alignment methods need to be chosen appropriately and carefully. The alignment method encapsulated in the class “aligner” in this pseudocode example is meant only to illustrate one simple approach to alignment. Many other alignment methods and techniques may be alternatively used for a conserved-element search. In certain embodiments of the present invention, no alignment is carried out, but, instead, all of the sequences to be analyzed are computationally cleaved into small subsequences that are analyzed to find conserved elements.

Alignment is carried out by the single public function member “align,” declared above on line 18. This function member takes two argument: (1) “orig,” a pointer to a set of sequences containing the sequences to be aligned; and (2) “aligned,” a pointer to an empty set of sequences that the alignment routine populates with aligned versions of the sequences in the set of sequences referenced by the argument “orig.” The alignment routine employs the private function members “findBest” and “score,” declared on lines 10-11, to identify the best average sequence from among the original sequences. The alignment routine then, in pairwise fashion, aligns each of the remaining sequences to this best sequence via the function member “pairwiseAlign,” declared on line 15. This “pairwiseAlign” function member calls the recursive function member “computeiRuns,” declared on line 14, to recursively align the next sequence to the reference sequence, or best sequence. In alignment, null characters, or gaps, may need to be inserted into either the reference sequence, via the private function member “insertNullsAllExcept,” or into the sequence currently being aligned via the private function member “insertNullsOnce.”

Next, the class “CE_Generator” is declared:

```
  1 class CE_Generator
  2 {
```

| | |
|---|---|
| 3 | private: |

| | |
|---|---|
| 4 | sequences* origSeqs; |
| 5 | sequences* alignedSeqs; |
| 6 | sequences conservedAAs; |

7

| | |
|---|---|
| 8 | compatibleAminoAcids* aa; |
| 9 | int numCAA; |
| 10 | positionAssignmentParameters* cd; |

11

| | |
|---|---|
| 12 | float table[numAminoAcids][maxPositionsPerSequence]; |
| 13 | posType map[maxPositionsPerSequence]; |
| 14 | int numC, numS; |
| 15 | Amino_Acid_Frequency list[maxFreqPerPos]; |
| 16 | int listNum; |
| 17 | int path[maxFreqPerPos]; |
| 18 | int pathNum; |

19

| 20 | void listClear( ); |
|---|---|
| 21 | void listAdd(float frequency, int aminoAicd); |
| 22 | void generateTable( ); |
| 23 | void clearTable( ); |
| 24 | bool compatible(int stkptr, int proposed); |
| 25 | bool contains(sequence* con, char* conee, int len); |
| 26 | bool varPos(int stkptr, double sum, int numV, double |
| | thresh, int pDepth); |
| 27 | void mapPos( ); |
| 28 | void enterCE (sequences* sqs, int i, int j, int end, |

| | |
|---|---|
| 29 | int depth, char* prevSeq); |

30

| | |
|---|---|
| 31 | public: |

| 32 | char get(int s, int i) {return (origSeqs−>get(s, i));}; |
|---|---|
| 33 | bool set(int s, int i, char val) {return (origSeqs−>set(s, i, |
| | val));}; |
| 34 | bool filter(sequences* sqs); |
| 35 | char aGet(int s, int i) {return (alignedSeqs−>get(s, i));}; |
| 36 | bool aSet(int s, int i, char val) {return (alignedSeqs−>set(s, |
| | i, val));}; |
| 37 | void getCEs(sequences* sqs, sequences* orig, sequences* |
| | aligned, |

| 38 | positionAssignmentParameters* c, |
|---|---|
| | compatibleAminoAcids* a, |
| 39 | int numCA); |

| | |
|---|---|
| 40 | CE_Generator( ); |

41 };

The class “CE_Generator” represents the conserved-element analysis logic that, in turn, represents one embodiment of the present invention. The class “CE_Generator” includes six public function members, declared above on lines 32-39: (1) “get,” a function member that returns the i^{th} original sequence; (2) “set,” a function member that allows the amino-acid identity for a position within the original sequences to be set; (3) “filter,” a function that allows for further processing of conserved elements, an implementation for which is not provided in the pseudocode; (4) “aGet,” a function member that retrieves the i^{th} alined sequence; (5) “aSet,” a function member that allows the amino acid at a particular position in a particular aligned sequence to be set; and (6) “getCEs,” the main function member of the class “CE_Generator” that is called to carry out a search for conserved elements within a set of sequences. The parameters to the public function member “getCEs” include: (1) “sqs,” a pointer to an instance of the class “sequences” that includes the identified conserved elements and that represents the results of a conserved-element search; (2) “orig,” a pointer to an instance of the class “sequences” that contains the original sequences to be analyzed for conserved elements; (3) “aligned,” a pointer to an instance of the class “sequences” that contains aligned versions of the original sequences; (4) “c,” an instance of the class “positionAssignmentParameters” that specifies the various parameter values that control the conserved-element search; (5) “a,” a pointer to an array of instances of the class “compatibleAminoAcids” which specify the allowed amino acid substitutions at variable positions within conserved elements; and (6) “numCA,” an integer value specifying the number of instances of the class “compatibleAminoAcids” in the array referenced by argument “a.”

Next, implementations for a number of the function members of the classes “compatibleAminoAcids,” “sequence,” and “sequences,” are provided. These implementations are quite straightforwardly implemented, and are not further described or annotated:

```
  1 compatibleAminoAcids::compatibleAminoAcids( )
  2 {
```

| | |
|---|---|
| 3 | for (int i = 0; i < numAminoAcids − 1; i++) aminoAcids[i] = |
| | false; |

```
  4 }
  1 sequence& sequence::operator = (sequence s)
  2 {
```

| | |
|---|---|
| 3 | len = s.getLen( ); |
| 4 | for (int i = 0; i < len; i++) |

| | |
|---|---|
| 5 | seq[i] = s.get(i); |

| | |
|---|---|
| 6 | return *this; |

```
  7 }
  1 char sequence::get(int i)
  2 {
```

| | |
|---|---|
| 3 | if (i >= 0 && i < len) return seq[i]; |
| 4 | else return NULL_CHAR; |

```
  5 }
  1 bool sequence::set(int i, char val)
  2 {
```

| | |
|---|---|
| 3 | if (i >= 0 && i < maxPositionsPerSequence) |
| 4 | { |

| | |
|---|---|
| 5 | seq[i] = val; |
| 6 | if (i >= len) len = i + 1; |
| 7 | return true; |

| | |
|---|---|
| 8 | } |
| 9 | return false; |

```
10 }
  1 bool sequence::set(char* s, int ln)
  2 {
```

| | |
|---|---|
| 3 | int i = 0; |

4

| | |
|---|---|
| 5 | if (ln > maxPositionsPerSequence) return false; |
| 6 | len = ln; |
| 7 | while (ln−−) seq[i++] = *s++; |
| 8 | return true; |

```
  9 }
  1 bool sequence::insertNull(int i, int j)
  2 {
```

| | |
|---|---|
| 3 | int k, m, n; |

4

| | |
|---|---|
| 5 | if (len + j > maxPositionsPerSequence) return false; |
| 6 | m = len + j − 1; |
| 7 | n = m − j; |
| 8 | while (n >= i) |

| | |
|---|---|
| 9 | seq[m−−] = seq[n−−]; |

| | |
|---|---|
| 10 | for (k = i; k < i + j; k++) seq[k] = NULL_CHAR; |
| 11 | len = len + j; |
| 12 | return true; |

```
13 }
  1 sequence::sequence( )
  2 {
```

| | |
|---|---|
| 3 | int i; |

4

| | |
|---|---|
| 5 | for (i = 0; i < maxPositionsPerSequence; i++) seq[i] = ‘.’; |
| 6 | len = 0; |

```
  7 }
  1 sequence& sequences::operator [ ] (int i)
  2 {
```

| | |
|---|---|
| 3 | if (i < maxNumSequences && i >= 0) |
| 4 | { |

| | |
|---|---|
| 5 | if (num < i + 1) num = i + 1; |
| 6 | return seqs[i]; |

| | |
|---|---|
| 7 | } |
| 8 | else return NULL_SEQ; |

```
  9 }
  1 sequence* sequences::getSeq(int i)
  2 {
```

| | |
|---|---|
| 3 | if (i < num && i >= 0) |

| | |
|---|---|
| 4 | return &(seqs[i]); |

| | |
|---|---|
| 5 | else return &(NULL_SEQ); |

```
  6 }
  1 bool sequences::addSeq(char* sq, int ln)
  2 {
```

| | |
|---|---|
| 3 | if (num < maxNumSequences − 1) |

| | |
|---|---|
| 4 | if (seqs[num].set(sq, ln)) |
| 5 | { |

| | |
|---|---|
| 6 | num++; |
| 7 | return true; |

| | |
|---|---|
| 8 | } |

| | |
|---|---|
| 9 | return false; |

```
10 }
  1 bool sequences::addSeq(sequence* sq)
  2 {
```

| | |
|---|---|
| 3 | if (num < maxNumSequences − 1) |
| 4 | { |

| | |
|---|---|
| 5 | seqs(num) = *sq; |
| 6 | num++; |
| 7 | return true; |

| | |
|---|---|
| 8 | } |
| 9 | return false; |

```
10 }
  1 int sequences::addSeq( )
  2 {
```

| | |
|---|---|
| 3 | if (num < maxNumSequences − 1) |

| | |
|---|---|
| 4 | num++; |

| | |
|---|---|
| 5 | return num − 1; |

```
  6 }
  1 bool sequences::setSeq(sequence* sq, int i)
  2 {
```

| | |
|---|---|
| 3 | if (i >= 0 && i < maxNumSequences) |
| 4 | { |

| | |
|---|---|
| 5 | seqs[i] = *sq; |
| 6 | if num < i + 1) |

| | |
|---|---|
| 7 | num = i + 1; |

| | |
|---|---|
| 8 | return true; |

| | |
|---|---|
| 9 | } |
| 10 | return false; |

```
11 }
  1 char sequences::get(int s, int i)
  2 {
```

| | |
|---|---|
| 3 | if (s < num && s >= 0) |

| | |
|---|---|
| 4 | return seqs[s].get(i); |

| | |
|---|---|
| 5 | else return NULL_CHAR; |

```
  6 }
  1 bool sequences::set(int s, int i, char val)
  2 {
```

| | |
|---|---|
| 3 | if (s < num && s >= 0) |

| | |
|---|---|
| 4 | if (seqs[s].set(i, val)) return true; |

| | |
|---|---|
| 5 | return NULL_CHAR; |

```
  6 }
  1 void sequences::clear( )
  2 {
```

| | |
|---|---|
| 3 | int i; |

4

| | |
|---|---|
| 5 | for (i = 0; i < maxNumSequences; i++) |

| | |
|---|---|
| 6 | seqs[i].setLen(0); |

| | |
|---|---|
| 7 | num = 0; |

```
  8 }
  1 sequences::sequences( )
  2 {
```

| | |
|---|---|
| 3 | num = 0; |

4 };

Next, implementations for function members of the class “aligner” are discussed. As mentioned above, there are a variety of different alignment methods and technologies that may be used for sequence alignment. The logic included in the class “aligner” is extremely simplistic and straightforward, but may provide adequate alignment in certain cases. It is included in the pseudocode for completeness and to illustrate an example of alignment, but is in no way intended to define or limit the present invention or the types of alignment techniques and methodologies that may be chosen for conserved-element analysis.

Implementations for the aligner function members “findBest” and “score” are next provided:

| 1 void aligner::findBest( ) | |
|---|---|
| 2 { | |
| 3 | int i, j; |
| 4 | double bestScore = 0; |
| 5 | double tScore; |
| 6 | |
| 7 | for (i = 0; i < origSeqs−>getNum( ); i++) |
| 8 | { |

| | |
|---|---|
| 9 | tScore = 0; |
| 10 | for (j = 0; j < origSeqs−>getNum( ); j++) |

| | |
|---|---|
| 11 | if (i != j) tScore += score(i,j); |

| | |
|---|---|
| 12 | if (tScore > bestScore) |
| 13 | { |

| | |
|---|---|
| 14 | bestScore = tScore; |
| 15 | best = i; |

| | |
|---|---|
| 16 | } |

| 17 | } |
|---|---|
| 18 } | |
| 1 double aligner::score(int i, int j) | |
| 2 { | |
| 3 | double res = 0.0; |
| 4 | sequence* p = origSeqs−>getSeq(i); |
| 5 | sequence* q = origSeqs−>getSeq(j); |
| 6 | int n; |
| 7 | |
| 8 | if (p−>getLen( ) > q−>getLen( )) n = q−>getLen( ) − 1; |
| 9 | else n = p−>getLen( ) − 1; |
| 10 | do |
| 11 | { |

| | |
|---|---|
| 12 | if (p−>get(n) == q−>get(n)) res += 1; |

| | |
|---|---|
| 13 | } while (n−−); |
| 14 | return res; |

15 }

The function member “score” simply computes the number of positions in two sequences, identified by the indexes i and j, which contain identical amino-acid identifiers. The function member “findBest” computes all possible pairwise scores among the set of original sequences, and selects, as the best sequence, the sequence with the best, or highest, cumulative score.

Next, an implementation for the function members “insertNullsOnce” and “insertNullsAllExcept” are provided:

```
  1 bool aligner::insertNullsOnce(int i, int j, int nm)
  2 {
```

| | |
|---|---|
| 3 | return ((*alignedSeqs)[i].insertNull(j, nm)); |

```
  4 }
  1 bool aligner::insertNullsAllExcept(int i, int j, int nm)
  2 {
```

| | |
|---|---|
| 3 | int k; |

4

| | |
|---|---|
| 5 | for (k = 0; k < i; k++) |

| | |
|---|---|
| 6 | if ((*alignedSeqs)[k].getLen( ) > 0) |

| | |
|---|---|
| 7 | if (!(*alignedSeqs)[k].insertNull(j, nm)) return false; |

| | |
|---|---|
| 8 | for (k = i + 1; k < alignedSeqs−>getNum( ); k++) |

| | |
|---|---|
| 9 | if ((*alignedSeqs)[k].getLen( ) > 0) |

| | |
|---|---|
| 10 | if (!(*alignedSeqs)[k].insertNull(j, nm)) return false; |

| | |
|---|---|
| 11 | return true; |

12 }

The function member “insertNullsOnce” inserts a null character at a specified position within the sequence that is being aligned. By contrast, the function member “insertNullsAllExcept” inserts null characters at the same position within the reference sequence, or best sequence, and all already aligned sequences. In certain cases, null characters are inserted into the sequence being currently aligned during the alignment process, while, in other cases, null characters are inserted into the reference, or best, sequence and all already aligned sequences.

Next, an implementation for the function member “computeIRuns” is provided:

```
  1 void aligner::computeIRuns(int iStart, int jStart, int iEnd, int jEnd, int
  ref, int s)
  2 {
```

| | |
|---|---|
| 3 | sequence& p = (*alignedSeqs)[ref]; |
| 4 | sequence& q = (*alignedSeqs)[s]; |
| 5 | int i, j, k, m, metric, n; |
| 6 | int iSz = iEnd − iStart + 1, jSz = jEnd − jStart + 1; |
| 7 | int szDiff, absDiff; |
| 8 | int bstM; |
| 9 | int diff, valid, bks; |

10

| | |
|---|---|
| 11 | bstM = −1; |
| 12 | bestSz = −1; |
| 13 | szDiff = iSz − jSz; |
| 14 | if (szDiff < 0) szDiff = −szDiff; |
| 15 | for (i = iStart; i <= iEnd; i++) |

| | |
|---|---|
| 16 | for (j = jStart; j <= jEnd; j++) |
| 17 | { |

| | |
|---|---|
| 18 | if ((jEnd − j + 1) < bestSz) break; |
| 19 | n = 0; |
| 20 | bks = 0; |
| 21 | k = i; |
| 22 | m = j; |
| 23 | while (p[k] == q[m]) |
| 24 | { |

| | |
|---|---|
| 25 | n++; |
| 26 | if (p[k] == NULL_CHAR) bks++; |
| 27 | k++; |
| 28 | m++; |
| 29 | if (k > iEnd ∥ m > jEnd) break; |

| | |
|---|---|
| 30 | } |
| 31 | diff = i − j; |
| 32 | if (diff < 0) diff = −diff; |
| 33 | valid = n − bks; |
| 34 | if (diff > szDiff) absDiff = diff − szDiff; |
| 35 | else absDiff = szDiff − diff; |
| 36 | metric = valid − absDiff; |
| 37 | if (valid > 0 && metric > bstM) |
| 38 | { |

| | |
|---|---|
| 39 | bestJ = j; |
| 40 | bestI = i; |
| 41 | bestSz = n; |
| 42 | bstM = metric; |

| | |
|---|---|
| 43 | } |

| | |
|---|---|
| 44 | } |

45 }

The function member “computeIRuns” attempts to find the longest string of amino-acids identifiers common to a currently considered portions of the reference sequence and a currently considered portion of a sequence currently being aligned to the reference sequence. In addition, the function member “computeIRuns” attempts to find a best-aligned common sequence of amino-acid identifiers. As the alignment between a run decreases, or the offset between the starting positions of the common run in the two sequences increases, the run is more greatly penalized. In the outer nested while-loops of the function member “computeiRuns,” beginning on lines 15 and 16, the function member “computeIRuns” tries all possible starting positions within the two sequences “s” and “ref” being compared and aligned. In the inner while-loop, on lines 23-30, pointers are iteratively advanced from the currently considered starting positions as long as the contents of the sequence positions referenced by the pointers in the two compared sequences contain the same amino-acid identifier. At the end of this while-loop, the size of any detected, commonly shared run of amino-acid identifiers is computed, along with the difference in alignment of the runs in the two sequences, or offset between starting positions of the commonly shared subsequence, and a metric is computed, on line 36, to balance length and alignment. If the value of the metric is better than the best metric so far computed, then a number of variables are set, on lines 39-42, to indicate that a best new commonly shared run of amino-acid identifiers, or commonly shared subsequence, has been found in the two sequences.

Next, an implementation of the function member “painviseAlign” is provided:

```
  1 void aligner::pairwiseAlign(int iStart, int jStart, int iEnd, int jEnd, int
  ref, int s)
  2 {
```

| | |
|---|---|
| 3 | int is, ie, js, je; |
| 4 | int iSz = iEnd − iStart; |
| 5 | int jSz = jEnd − jStart; |

6

| | |
|---|---|
| 7 | computeIRuns(iStart, jStart, iEnd, jEnd, ref, s); |
| 8 | if (bestSz < 0) |
| 9 | { |

| | |
|---|---|
| 10 | if (iSz < jSz) insertNullsAllExcept(s, iStart, jSz − iSz); |
| 11 | else if (jSz < iSz) insertNullsOnce(s, jStart, iSz − jSz); |
| 12 | return; |

| | |
|---|---|
| 13 | } |

14

| | |
|---|---|
| 15 | is = bestI; |
| 16 | ie = bestI + bestSz; |
| 17 | js = bestJ; |
| 18 | je = bestJ + bestSz; |

19

| | |
|---|---|
| 20 | pairwiseAlign(ie, je, iEnd, jEnd, ref, s); |
| 21 | pairwiseAlign(iStart, jStart, is − 1, js − 1, ref, s); |

22 }

This function member recursively aligns the sequence specified by index “s” to the reference, or best, sequence identified by index “ref.” On line 7, the function member “pairwiseAlign” calls the function member “computeIRuns” to find the best length of matching identical amino-acid identifiers in the two sequences, and then recursively calls itself, on lines 20 and 21, to align portions of the two sequences following and prior to the identified best run.

Next, an implementation of the function member “align” is provided:

```
  1 void aligner::align(sequences* orig, sequences* aligned)
  2 {
```

| | |
|---|---|
| 3 | int i, num = orig−>getNum( ); |
| 4 | origSeqs = orig; |
| 5 | alignedSeqs = aligned; |

6

| | |
|---|---|
| 7 | findBest( ); |
| 8 | alignedSeqs−>clear( ); |

9

| | |
|---|---|
| 10 | (*alignedSeqs)[best] = *(origSeqs−>getSeq(best)); |
| 11 | for (i = 0; i < best; i++) |
| 12 | { |

| 13 | (*alignedSeqs)[i] = *(origSeqs−>getSeq(i)); |
|---|---|
| 14 | pairwiseAlign(0, 0, |
| | alignedSeqs−>getSeq(best)−>getLen( ) − 1, |

| | |
|---|---|
| 15 | alignedSeqs−>getSeq(i)−>getLen( ) − 1, best, i); |

| | |
|---|---|
| 16 | } |
| 17 | for (i = best + 1; i < num; i++) |
| 18 | { |

| 19 | (*alignedSeqs)[i] = *(origSeqs−>getSeq(i)); |
|---|---|
| 20 | pairwiseAlign(0, 0, |
| | alignedSeqs−>getSeq(best)−>getLen( ) − 1, |

| | |
|---|---|
| 21 | alignedSeqs−>getSeq(i)−>getLen( ) − 1, best, i); |

| | |
|---|---|
| 22 | } |

23

The function member “align” determines the reference, or best, sequence, on line 7, via a call to the function member “findBest,” and then proceeds to align all sequences in the set of original sequences prior to the reference sequence, in the for-loop of lines 11-16, and then aligns all the sequences following the reference sequence in the for-loop of lines 17-22.

Next, implementations for function members of the class “CE_Generator” are provided. No implementation is provided for the function member “filter,” which is intended to illustrate that, following initial identification of conserved elements, additional considerations may be employed to discard certain of the identified conserved elements for various criteria. For example, initially identified conserved elements may be compared to host sequences in order to eliminate conserved elements similar or identical to native host sequences that, if included in a vaccine polymer, might elicit an autoimmune response. As another example, conserved elements that are known to be strongly immunodominant, and less than optimally effective in eliciting a desired, protective immune response, may also be eliminated or somehow identified for special positioning or inclusion at a special multiplicity within the vaccine. Other considerations may also be applied by the filter function. No implementation is provided for this function because the implementation generally depends on extraneous databases and other information, accessible through specialized interfaces that are beyond the scope of the present discussion, and may also be vaccine-type and host-type dependent.

Next, implementations with function members “clearTable” and “generateTable” are provided:

| 1 void CE_Generator::clearTable( ) | |
|---|---|
| 2 { | |
| 3 | int i, j; |
| 4 | |
| 5 | for (i = 0; i < numAminoAcids; i++) |

| | |
|---|---|
| 6 | for (j = 0; j < maxPositionsPerSequence; j++) |

| | |
|---|---|
| 7 | table[i][j] = 0; |

| 8 } | |
|---|---|
| 1 void CE_Generator::generateTable( ) | |
| 2 { | |
| 3 | int i, j; |
| 4 | |
| 5 | numC = (*alignedSeqs)[0].getLen( ); |
| 6 | numS = origSeqs−>getNum( ); |
| 7 | |
| 8 | clearTable( ); |
| 9 | for (i = 0; i < numS; i++) |

| | |
|---|---|
| 10 | for (j = 0; j < numC; j++) |

| | |
|---|---|
| 11 | table[aGet(i,j) − ‘a’][j]++; |

| | |
|---|---|
| 12 | for (i = 0; i < numAminoAcids; i++) |

| | |
|---|---|
| 13 | for (j = 0; j < numC; j++) |

| | |
|---|---|
| 14 | table[i][j] /= numS; |

15 }

These function members initialize and generate a table that includes the amino-acid-occurrence frequencies at each position within the set of aligned sequences. In other words, the table is a matrix of amino-acid-frequency of occurrence with respect to sequence position, with one axis, or index, spanning the possible amino acids, and another axis, or index, spanning all of the positions within the aligned set of sequences. Note that, after alignment, all aligned sequences have equal length. The frequencies range from 0 to 1, and are floating-point values computed by dividing the number of occurrences of each amino acid at each position by the total number of sequences, on line 14 of the function member “generateTable.” Again, as with many aspects of the pseudocode implementation, many different design choices and alternative algorithms are possible. For example, frequencies might be adjusted downward in the case that a position is only sparsely populated or, in other words, the null character is frequently observed at the position.

Next, implementations for the CE_Generator member functions “listClear” and “listAdd” are provided:

```
  1 void CE_Generator::listClear( )
  2 {
```

| | |
|---|---|
| 3 | int i; |

4

| | |
|---|---|
| 5 | for (i = 0; i < maxFreqPerPos; i++) |

| | |
|---|---|
| 6 | list[i].freq = 0; |

| | |
|---|---|
| 7 | listNum = 0; |

```
  8 }
  1 void CE_Generator::listAdd(float frequency, int aminoAicd)
  2 {
```

| | |
|---|---|
| 3 | int i, j; |

4

| | |
|---|---|
| 5 | if (listNum == 0) |
| 6 | { |

| | |
|---|---|
| 7 | list[0].freq = frequency; |
| 8 | list[0].amino_acid = aminoAicd; |
| 9 | listNum = 1; |
| 10 | return; |

| | |
|---|---|
| 11 | } |
| 12 | for (i = 0; i < listNum; i++) |

| | |
|---|---|
| 13 | if (frequency > list(i).freq) |
| 14 | { |

| | |
|---|---|
| 15 | j = listNum; |
| 16 | if (j == maxFreqPerPos) j = maxFreqPerPos − 1; |
| 17 | while (j > i) |
| 18 | { |

| | |
|---|---|
| 19 | list[j] = list[j − 1]; |
| 20 | j−−; |

| | |
|---|---|
| 21 | } |
| 22 | list[i].freq = frequency; |
| 23 | list[i].amino_acid = aminoAicd; |
| 24 | if (listNum < maxFreqPerPos) listNum++; |
| 25 | return; |

| | |
|---|---|
| 26 | } |

| | |
|---|---|
| 27 | if (listNum < maxFreqPerPos) |
| 28 | { |

| | |
|---|---|
| 29 | list[listNum].freq = frequency; |
| 30 | list[listNum].amino_acid = aminoAicd; |
| 31 | listNum++; |

| | |
|---|---|
| 32 | } |

33 }

The list that is created using these routines is a list of amino acid occurrences at a particular position within the aligned sequences. A list is created for each position, with the ten most frequent occurring amino acids, if ten or more amino acids occur at that position, maintained in the list in order of decreasing frequency of occurrence. This list is used to determine whether a position is a variable position and, if so, to determine a minimal set of amino acids with a combined frequency of occurrence greater than the variable threshold.

Next, an implementation for the CE_Generator function member “compatible” is provided:

```
  1 bool CE_Generator::compatible(int stkptr, int proposed)
  2 {
```

| | |
|---|---|
| 3 | int i, k; |
| 4 | bool res = true; |

5

| | |
|---|---|
| 6 | compatibleAminoAcids* ptr = aa; |

7

| | |
|---|---|
| 8 | for (i = 0; i < numCAA; i++) |
| 9 | { |

| | |
|---|---|
| 10 | res = true; |
| 11 | for (k = 0; k < stkptr; k++) |

| | |
|---|---|
| 12 | if (!ptr−>in(list[i].amino_acid + ‘a’)) |
| 13 | { |

| | |
|---|---|
| 14 | res = false; |
| 15 | break; |

| | |
|---|---|
| 16 | } |

| | |
|---|---|
| 17 | if (res) res = ptr−>in(list[proposed].amino_acid + ‘a’); |
| 18 | if (res) return true; |
| 19 | ptr++; |

| | |
|---|---|
| 20 | } |
| 21 | return false; |

22 }

The function member “compatible” determines whether an amino acid proposed to be included in the set of amino acids that together comprise a variable position is compatible with the other amino acids already included in the variable position.

Next, an implementation for the CE_Generator function member “varPos” is provided:

```
  1 bool CE_Generator::varPos(int stkptr, double sum, int numV, double
  thresh,
```

| | |
|---|---|
| 2 | int pDepth) |

3 {

| | |
|---|---|
| 4 | int i; |

5

| | |
|---|---|
| 6 | for (i = stkptr; i < listNum; i++) |

| | |
|---|---|
| 7 | { |
| 8 | if (pDepth == 0) pathNum = 0; |
| 9 | path[pDepth] = i; |
| 10 | if (compatible(stkptr, i)) |
| 11 | { |

| | |
|---|---|
| 12 | if (list[i].freq + sum >= thresh) |
| 13 | { |

| | |
|---|---|
| 14 | pathNum = pDepth; |
| 15 | return true; |

| | |
|---|---|
| 16 | } |
| 17 | else |
| 18 | { |

| 19 | if (numV == 1) continue; |
|---|---|
| 20 | if (varPos((stkptr + 1), sum + list[i].freq, numV − |
| | 1, thresh, |

| | |
|---|---|
| 21 | pDepth + 1)) |
| 22 | return true; |

| | |
|---|---|
| 23 | } |

| | |
|---|---|
| 24 | } |

| | |
|---|---|
| 25 | } |
| 26 | return false; |

27 }

The function member “varPos” recursively examines the ordered list of amino-acid frequencies prepared for a particular position in the aligned sequences to determine if there is a set of amino acids sized less than or equal to the maximum number of amino acids allotted a variable position with a combined frequency of occurrence greater than or equal to the threshold frequency of occurrence for a variable position. This function member returns a Boolean result indicating whether or not a particular position within the aligned sequences is a variable position.

Next, an implementation of the function member “mapPos” is provided:

```
  1 void CE_Generator::mapPos( )
  2 {
```

| | |
|---|---|
| 3 | int i, j, k; |

4

| | |
|---|---|
| 5 | for (j = 0; j < numC; j++) |
| 6 | { |

| | |
|---|---|
| 7 | listClear( ); |
| 8 | for (i = 0; i < numAminoAcids − 1; i++) |

| | |
|---|---|
| 9 | if (table[i][j] > 0) |

| | |
|---|---|
| 10 | listAdd(table[i][j], i); |

| | |
|---|---|
| 11 | if (list[0].freq > cd−>getConservedThreshold( )) |
| 12 | { |

| | |
|---|---|
| 13 | map[j] = conserved; |
| 14 | conservedAAs[0].set(j, list[0].amino_acid + ‘a’); |

| | |
|---|---|
| 15 | } |
| 16 | else if (varPos(0, 0, cd−>getMaxAAsAtVariablePosition( ), |

| | |
|---|---|
| 17 | cd−>getVariableThreshhold( ), 0)) |

| | |
|---|---|
| 18 | { |

| | |
|---|---|
| 19 | map[j] = variable; |
| 20 | for (k = 0; k <= pathNum; k++) |
| 21 | { |

| 22 | conservedAAs[k].set(j, |
|---|---|
| | list[path[k]].amino_acid + ‘a’); |

| | |
|---|---|
| 23 | } |
| 24 | conservedAAs[k).set(j, NULL_CHAR); |

| | |
|---|---|
| 25 | } |
| 26 | else map[j] = unconserved; |

| | |
|---|---|
| 27 | } |

28 }

The function member “mapPos” creates a one-dimensional map of the aligned sequence positions, for each position indicating when the position is conserved, variable, or unconserved. For variable positions, identities of the amino acids at those positions are preserved in an instance of the class “sequences,” “conservedAAs.”

Next, implementation of the CE_Generator function member “contains” is provided:

```
  1 bool CE_Generator::contains(sequence* con, char* conee, int len)
  2 {
```

| | |
|---|---|
| 3 | int i, j, k; |
| 4 | bool res; |

5

| | |
|---|---|
| 6 | for (i = 0; i <= (con−>getLen( ) − len); i++) |
| 7 | { |

| | |
|---|---|
| 8 | res = true; |
| 9 | k = i; |
| 10 | for (j = 0; j < len; j++) |
| 11 | { |

| | |
|---|---|
| 12 | if (conee[j] != con−>get(k++)) |
| 13 | { |

| | |
|---|---|
| 14 | res = false; |
| 15 | break; |

| | |
|---|---|
| 16 | } |

| | |
|---|---|
| 17 | } |
| 18 | if (res == true) return true; |

| | |
|---|---|
| 19 | } |
| 20 | return false; |

21 }

The function member “contains” determines whether a conserved element identified during conserved-element analysis has already been included in a set of conserved elements already found during the conserved-element analysis.

Next, an implementation of the function member “enterCE” is provided:

```
  1 void CE_Generator::enterCE (sequences* sqs, int i, int j, int end, int
  depth,
```

| | |
|---|---|
| 2 | char* prevSeq) |

3 {

| | |
|---|---|
| 4 | char tseq[maxPositionsPerSequence]; |
| 5 | int k, t; |
| 6 | bool already; |

7

| | |
|---|---|
| 8 | if (depth > 0) |
| 9 | { |

| | |
|---|---|
| 10 | if (conservedAAs[depth].get(j) == NULL_CHAR) return; |
| 11 | for (k = i, t = 0; k < j; k++, t++) tseq[t] = prevSeq[t]; |
| 12 | tseq[t++] = conservedAAs[depth].get(j++); |
| 13 | enterCE(sqs, i, j, end, depth + 1, tseq); |

| | |
|---|---|
| 14 | } |
| 15 | else t = j − i; |
| 16 | while (j < end) |
| 17 | { |

| | |
|---|---|
| 18 | if (map[j] == variable) |

| | |
|---|---|
| 19 | enterCE(sqs, i, j, end, depth + 1, tseq); |

| | |
|---|---|
| 20 | tseq[t++] = conservedAAs[0].get(j++); |

| | |
|---|---|
| 21 | } |
| 22 | already = false; |
| 23 | for (k = 0; k < sqs−>getNum( ); k++) |

| | |
|---|---|
| 24 | if (contains(sqs−>getSeq(k), tseq, j − i)) |
| 25 | { |

| | |
|---|---|
| 26 | already = true; |
| 27 | break; |

| | |
|---|---|
| 28 | } |

| | |
|---|---|
| 29 | if ((already) sqs−>addSeq(tseq, j − i); |

30 }

The function member “enterCE” enters a next identified conserved element into the set of conserved elements that represents the result of conserved-element analysis. When the next identified conserved element includes one or more variable positions, all possible related sequences, obtained by substitution of the various amino acids that occur at the variable positions, are generated and entered.

Next, an implementation for the CE_Generator function member “getCEs” is provided:

```
  1 void CE_Generator::getCEs(sequences* sqs, sequences* orig,
  sequences*
```

| | |
|---|---|
| 2 | aligned, positionAssignmentParameters* c, |
| 3 | compatibleAminoAcids* a, int numCA) |

4 {

| | |
|---|---|
| 5 | int i, j; |
| 6 | int numV, len; |

7

| | |
|---|---|
| 8 | origSeqs = orig; |
| 9 | alignedSeqs = aligned; |

10

| | |
|---|---|
| 11 | generateTable( ); |

12

| 13 | numC = (*alignedSeqs)[0].getLen( ), numS = |
|---|---|
| | origSeqs−>getNum( ); |

14

| | |
|---|---|
| 15 | aa = a; |
| 16 | cd = c; |
| 17 | numCAA = numCA; |

18

| | |
|---|---|
| 19 | mapPos( ); |

20

| | |
|---|---|
| 21 | for (i = 0; i < numC; i++) |
| 22 | { |

| | |
|---|---|
| 23 | numV = 0; |
| 24 | for (j = i; j < numC; j++) |
| 25 | { |

| | |
|---|---|
| 26 | if (map[j] == unconserved) break; |
| 27 | if (map[j] == variable) numV++; |
| 28 | if (numV > cd−>getMaxVariablePositions( )) break; |

| | |
|---|---|
| 29 | } |
| 30 | len = j − i; |
| 31 | if (len >= cd−>getThresholdCELength( )) |

| | |
|---|---|
| 32 | enterCE(sqs, i, i, j, 0, NULL); |

| | |
|---|---|
| 33 | } |

34 }

This is the main function member of the class “CE_Generator.” First, on line 11, the table of amino-acid-occurrence frequencies is generated. Then, on line 19, the one-dimensional map of the aligned-sequence positions, indicating whether each position is conserved, variable, or unconserved, is generated via a call to the function member “mapPos.” Finally, in the for-loop of lines 21-34, the one-dimensional map is exhaustively searched for conserved elements that meet all of the thresholds and parameters, including the length threshold, number of variable positions threshold, number of amino acids allowed at a variable position threshold, and other parameters. Each identified conserved element not already entered into the results set is entered into the results set via a call to “enterCE” on line 32.

Finally, a truncated version of an exemplary program for searching a set of sequences for conserved elements is provided:

| 1int main(int argc, char* argv[ ]) | |
|---|---|
| 2{ | |
| 3 | sequences orig, aligned, reslt; |
| 4 | aligner align; |
| 5 | CE_Generator ce; |
| 6 | |
| 7 | compatibleAminoAcids cmpaa[4]; |
| 8 | positionAssignmentParameters c; |
| 9 | |
| 10 | c.setThresholdCELength(4); |
| 11 | c.setMaxVariablePositions(1); |
| 12 | c.setConservedThreshold(0.85); |
| 13 | c.setVariableThreshhold(0.8); |
| 14 | c.setMaxAAsAtVariablePosition(2); |
| 15 | |
| 16 | align.align(&orig, &aligned); |
| 17 | ce.getCEs(&reslt, &orig, &aligned, &c, cmpaa, 1); |
| 18 | |
| 19 | return 0; |

20 }

In an actual program, sequences are added to an instance of class “sequences,” “orig,” through calls to the sequences function member “addSeq,” and compatible sets of amino acids are similarly added to an instance of the class “compatibleAminoAcids.”

Again, there are an essentially unlimited number of different implementations of the conserved-element analysis logic that represent embodiments of the present invention. There are many different design choices, additional parameters and constraints that may be considered, different analytical techniques with different computational efficiencies, that may all be considered when addressing particular problem domains, including particular types of vaccines, particular hosts, and particular pathogens. For example, vaccines may be targeted to eukaryotic parasite pathogens, bacterial pathogens, and complex viral pathogens, with much larger genomes and corresponding proteomes than HIV, perhaps requiring different computational strategies and additional criteria for selecting conserved elements. Certain of the above-described features of the C++-like pseudocode may be omitted, without significantly impacting conserved-element analysis.

A Perl program actually used for generating conserved sequences for the HIV virus is provided in FIG. 11. The Perl program does not include alignment, but depends on input sequences having been aligned by another program or routine,

In addition, as suggested above, various embodiments of the present invention may avoid aligning sequences altogether. Instead, the set of sequences to be analyzed may be decomposed computationally into small subsequences that are then computationally re-assembled to identify conserved elements. Many other computational approaches are also possible.

Application of the above-described method for selecting conserved elements (“CEs”) from aligned sequences has produced a set of CE peptide sequences with very high conservation and a set with slightly less conservation from large sets of aligned HIV gene sequences. The analysis was done on a gene-by-gene basis, using the following numbers of HIV-gene variants: (1) gag—619; (2) pol—615; (3) vif—967; (4) vpr—835; (5) tat—1225; (6) rev—938; (7) vpu—925; (8) env—871; (9) nef—1474. Highly conserved CEs are included below in Table 1:

TABLE 1
Highly Conserved HIV pol;ypeptide CEs.

| Gene | | |
|---|---|---|
| Product | Sequence | SEQ ID |
| Gag | PRTLNAWVKVIEEK | SEQ ID No. 1 |
| Gag | PRTLNAWVKVVEEK | SEQ ID No. 2 |
| Gag | ARTLNAWVKVIEEK | SEQ ID No. 3 |
| Gag | ARTLNAWVKVVEEK | SEQ ID No. 4 |
| Gag | MLNTVGGHQAAMQ | SEQ ID No. 5 |
| Gag | MLNIVGGHQAAMQ | SEQ ID No. 6 |
| Gag | REPRGSDIAG | SEQ ID No. 7 |
| Gag | RDPRGSDIAG | SEQ ID No. 8 |
| Gag | LGLNKIVRMYSP | SEQ ID No. 9 |
| Gag | MGLNKIVRMYSP | SEQ ID No. 10 |
| Gag | SILDIRQGPKEPFRDYVDRF | SEQ ID No. 11 |
| Gag | SILDIRQGPKEPFRDYVDRF | SEQ ID No. 12 |
| Gag | SILDIRQGPKEPFRDYVDRF | SEQ ID No. 13 |
| Gag | SILDIKQGPKESFRDYVDRF | SEQ ID No. 14 |
| Gag | EEMMTACQGVGGP | SEQ ID No. 15 |
| Gag | EEMMSACQGVGGP | SEQ ID No. 16 |
| Pol | PQITLWQRP | SEQ ID No. 17 |
| Pol | EALLDTGADDTV | SEQ ID No. 18 |
| PoI | MIGGIGGFIKV | SEQ ID No. 19 |
| Pol | GCTLNFPISP | SEQ ID No. 20 |
| Pol | LKPGMDGP | SEQ ID No. 21 |
| Pol | IGPENPYNTP | SEQ ID No. 22 |
| Pol | WRKLVDFRELNK | SEQ ID No. 23 |
| PoI | TQDFWEVQLGIPHP | SEQ ID No. 24 |
| Pol | SVTVLDVGDAYFS | SEQ ID No. 25 |
| Pol | FRKYTAFTIPS | SEQ ID No. 26 |
| Pol | RYQYNVLPQGWKGSP | SEQ ID No. 27 |
| PoI | DDLYVGSDL | SEQ ID No. 28 |
| Pol | KHQKEPPFLWMGYELHPD | SEQ ID No. 29 |
| Pol | WTVNDIQKLVGKLNWASQIY | SEQ ID No. 30 |
| Pol | EAELELAENREIL | SEQ ID No. 31 |
| Pol | QWTYQIYQE | SEQ ID No. 32 |
| Pol | KNLKTGKYA | SEQ ID No. 33 |
| Pol | YWQATWIP | SEQ ID No. 34 |
| Pol | NTPPLVKLWY | SEQ ID No. 35 |
| Pol | VNIVTDSQY | SEQ ID No. 36 |
| Pol | WVPAHKGIGGNELDCTHLEGK | SEQ ID No. 37 |
| Pol | LDCTHLEGK | SEQ ID No. 38 |
| Pol | VAVHVASGY | SEQ ID No. 39 |
| Pol | LKLAGRWPV | SEQ ID No. 40 |
| Pol | GIPYNPQSQGV | SEQ ID No. 41 |
| Pol | TAVQMAVFIHNFKR | SEQ ID No. 42 |
| Pol | WKGPAKLLWKGEGAVV | SEQ ID No. 43 |
| Env | WVTVYYGVPVW | SEQ ID No. 44 |
| Env | WATHACVPTDP | SEQ ID No. 45 |
| Env | STQLLLNGS | SEQ ID No. 46 |
| Env | LTVWGIKQLQ | SEQ ID No. 47 |
| Vif | IVWQVDRMRI | SEQ ID No. 48 |

An additional set of less highly conserved CE peptide sequences has been identified from large sets of aligned HIV gene sequences by relaxing certain of the threshold constraints:

TABLE 2
Less Highly Conserved HIV polypeptide CEs.

| Gene | | |
|---|---|---|
| Product | Sequence | SEQ ID |
| Gag | ALSEGATP | SEQ ID No. 49 |
| Gag | ALAEGATP | SEQ ID No. 50 |
| Gag | HKARVLAE | SEQ ID No. 51 |
| Gag | HKARILAE | SEQ ID No. 52 |
| Gag | APRKKGCWAMS | SEQ ID No. 53 |
| Gag | APRKRGCWAMS | SEQ ID No. 54 |
| Gag | EGHQMKDCKCG | SEQ ID No. 55 |
| Gag | EGHQMKECKCG | SEQ ID No. 56 |
| Env | HNVWATHACVPTDP | SEQ ID No. 57 |
| Env | HNIWATHACVPTDP | SEQ ID No. 58 |
| Env | VQCTHGIKPVVSTQLLLNGS | SEQ ID No. 59 |
| Env | VQCTHGIKPVISTQLLLNGS | SEQ ID No. 60 |
| Env | VQCTHGIRPVVSTQLLLNGS | SEQ ID No. 61 |
| Env | VQCTHGIRPVISTQLLLNGS | SEQ ID No. 62 |
| Env | LTVWGIKQLQAR | SEQ ID No. 63 |
| Env | LTVWGIKQLQAR | SEQ ID No. 64 |
| Rev | RNRRRRWR | SEQ ID No. 65 |
| Rev | KNRRRRWR | SEQ ID No. 66 |
| Vif | IVWQVDRMKI | SEQ ID No. 67 |
| Vif | VGSLQYLAL | SEQ ID No. 68 |

Alternative embodiments of the conserved-element identifying methods of the present invention may produce additional conserved elements. In addition, analysis of a greater number of HIV sequences from additional strains may lead to modification of the final set of conserved elements for HIV.

Once conserved elements are identified, they are used to construct one or more biopolymers used directly as a vaccine, or used in intermediate steps of vaccine development. The combination of conserved elements to produce vaccine biopolymers, or intermediate biopolymers used to produce vaccines, is a complex process that may involve many considerations, constraints, and use of linker sequences and other sequences in addition to the conserved elements. The problem of combining CEs to produce vaccine-relate biopolymers may be parameterized, just as CE-identification methods are parameterized. For example, the problem of combining CEs to produce a vaccine-relate biopolymer may optimize variables, including the number of copies of each CE to include in the biopolymer, the relative positions of CEs, the length and types of linker sequences used to join the CEs together, the number of discrete biopolymers to use for the vaccine, or as intermediate biopolymers, and other such parameters. Optimization constraints and goals may include the frequency of display of CEs by antigen-presenting cells, the effective concentration, or copy number, of displayed CEs, the effectiveness of the immune response elicited by the vaccine, and other such constraints and goals, avoiding inadvertent generation of undesirable sequence fragments displayed by antigen-presenting cells, overall size constraints for a useable vaccine biopolymer, and other such constraints.

Although the present invention has been described in terms of particular embodiments, it is not intended that the invention be limited to these embodiments. Modifications within the spirit of the invention will be apparent to those skilled in the art. For example, it should be noted that, although certain embodiments of the present invention are described for identifying conserved elements of viral proteomes, alternative method embodiments may be directed to identifying conserved viral RNA subsequences or vDNA subsequences, and designing CEVacs based on conserved viral RNA subsequences or vDNA subsequences. As discussed above, any of a vast number of different subsequence-selection criteria may be applied in order to identify conserved elements. Once the conserved elements within the two-dimensional viral proteome array, discussed with reference to FIG. 8, are identified, various techniques are used to select entire conserved subsequences, or portions of conserved subsequences, for incorporation into expression vectors in order to produce a synthetic vaccine according to the present invention. In the above-described embodiment, no unconserved amino-acids are allowed in conserved elements, but, in alternative embodiments, a small, maximum number of unconserved variable positions may be allowed. Although the above-described conserved-element-based vaccine design methods are not specifically designed to elicit a humoral immune response, conserved-element vaccines may indeed elicit antibody production and an antibody-mediated immune response to a target pathogen. For example, conserved elements identified within the HIV env gene may effectively elicit humoral immune response.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for identifying conserved elements in a set of biopolymer sequences for incorporation into a vaccine, each sequence comprising an ordered set of positions and each position containing an identifier of a biopolymer monomer, the method comprising:
classifying each position within the biopolymer sequences as conserved, variable, or unconserved; and
selecting from the biopolymer sequences a set of subsequences, each having a length, in positions, greater than a threshold value, less than a threshold number of variable positions, and less than a threshold number of unconserved positions.

2. The method of claim 1 wherein a conserved position is a position at which a single monomer occurs at greater than a threshold frequency over the entire set of biopolymer sequences.

3. The method of claim 1 wherein a variable position is a position at which a number of monomers less than a threshold number of monomers occur at greater than a threshold frequency over the entire set of biopolymer sequences.

4. The method of claim 1 wherein an unconserved position is a position that is neither conserved nor variable.

5. The method of claim 1 further including filtering the selected set of subsequences to remove subsequences that, based on additional criteria, are not suitable for incorporation into a vaccine.

6. The method of claim 5 wherein the additional criteria include:
similarity or identity with host subsequences; and
an indication that the subsequence is immunodominant.

7. The method of claim 1 wherein the selected subsequences, optionally filtered to remove immunodominant subsequences and subsequences that have greater than a threshold similarity with respect to a host biopolymer, are incorporated into one or more biopolymers used as a vaccine.

8. The method of claim 1 wherein the biopolymer sequences are selected from among:
polypeptide sequences;
RNA sequences; and
DNA sequences.

9. The method of claim 1 wherein the threshold number of unconserved positions is 1.

10. The method of claim 1 further including, prior to classifying each position within the biopolymer sequences as conserved, variable, or unconserved:
aligning the biopolymer sequences in the set of biopolymer sequences with one another.

11. An HIV vaccine polypeptide comprising at least one copy of at least 80% of the following conserved-element peptide sequences, with 10% of the total peptide subsequences of the HIV vaccine polypeptide corresponding to HIV proteome peptide fragments not listed below:

| | |
|---|---|
| PRTLNAWVKVIEEK,; | SEQ ID No. 1 |
| PRTLNAWVKVVEEK,; | SEQ ID No. 2 |
| ARTLNAWVKVIEEK,; | SEQ ID No. 3 |
| ARTLNAWVKVVEEK,; | SEQ ID No. 4 |
| MLNTVGGHQAAMQ,; | SEQ ID No. 5 |
| MLNIVGGHQAAMQ,; | SEQ ID No. 6 |
| REPRGSDIAG,; | SEQ ID No. 7 |
| RDPRGSDIAG,; | SEQ ID No. 8 |
| LGLNKIVRMYSP,; | SEQ ID No. 9 |
| MGLNKIVRMYSP,; | SEQ ID No. 10 |
| SILDIRQGPKEPFRDYVDRF,; | SEQ ID No. 11 |
| SILDIRQGPKESFRDYVDRF,; | SEQ ID No. 12 |
| SILDIKQGPKEPFRDYVDRF,; | SEQ ID No. 13 |
| SILDIKQGPKESFRDYVDRF,; | SEQ ID No. 14 |
| EEMMTACQGVGGP,; | SEQ ID No. 15 |
| EEMMSACQGVGGP,; | SEQ ID No. 16 |
| PQITLWQRP,; | SEQ ID No. 17 |
| EALLDTGADDTV,; | SEQ ID No. 18 |
| MIGGIGGFIKV,; | SEQ ID No. 19 |
| GCTLNFPISP,; | SEQ ID No. 20 |
| LKPGMDGP,; | SEQ ID No. 21 |
| IGPENPYNTP,; | SEQ ID No. 22 |
| WRKLVDFRELNK,; | SEQ ID No. 23 |
| TQDFWEVQLGIPHP,; | SEQ ID No. 24 |
| SVTVLDVGDAYFS,; | SEQ ID No. 25 |
| FRKYTAFTIPS,; | SEQ ID No. 26 |
| RYQYNVLPQGWKGSP,; | SEQ ID No. 27 |
| DDLYVGSDL,; | SEQ ID No. 28 |
| KHQKEPPFLWMGYELHPD,; | SEQ ID No. 29 |
| WTVNDIQKLVGKLNWASQIY,; | SEQ ID No. 30 |
| EAELELAENREIL,; | SEQ ID No. 31 |
| QWTYQIYQE,; | SEQ ID No. 32 |
| KNLKTGKYA,; | SEQ ID No. 33 |
| YWQATWIP,; | SEQ ID No. 34 |
| NTPPLVKLWY,; | SEQ ID No. 35 |
| VNIVTDSQY,; | SEQ ID No. 36 |
| WVPAHKGIGGNELDCTHLEGK,; | SEQ ID No. 37 |
| LDCTHLEGK,; | SEQ ID No. 38 |
| VAVHVASGY,; | SEQ ID No. 39 |
| LKLAGRWPV,; | SEQ ID No. 40 |
| GIPYNPQSQGV,; | SEQ ID No. 41 |
| TAVQMAVFIHNFKR,; | SEQ ID No. 42 |
| WKGPAKLLWKGEGAVV,; | SEQ ID No. 43 |
| WVTVYYGVPVW,; | SEQ ID No. 44 |
| WATHACVPTDP,; | SEQ ID No. 45 |
| STQLLLNGS,; | SEQ ID No. 46 |
| LTVWGIKQLQ,; | SEQ ID No. 47 |
| and | |
| IVWQVDRMRI,. | SEQ ID No. 48 |

12. The HIV vaccine polypeptide of claim 11 comprising at least one copy of at least 90% of the conserved-element peptide sequences.

13. The HIV vaccine polypeptide of claim 11 comprising at least one copy of at least 95% of the conserved-element peptide sequences.

14. An HIV vaccine DNA encoding the HIV vaccine polypeptide of claim 11.

15. The HIV vaccine polypeptide of claim 11 including no HIV proteome peptide fragments not listed in claim 11.

16. An HIV vaccine polypeptide comprising at least one copy of at least 80% of the following conserved-element peptide sequences, with 10% of the total peptide subsequences of the HIV vaccine polypeptide corresponding to HIV proteome peptide fragments not listed below:

| | |
|---|---|
| PRTLNAWVKVIEEK,; | SEQ ID No. l |
| PRTLNAWVKVVEEK,; | SEQ ID No. 2 |
| ARTLNAWVKVIEEK,; | SEQ ID No. 3 |
| ARTLNAWVKVVEEK,; | SEQ ID No. 4 |
| MLNTVGGHQAAMQ,; | SEQ ID No. 5 |
| MLNIVGGHQAAMQ,; | SEQ ID No. 6 |
| REPRGSDIAG,; | SEQ ID No. 7 |
| RDPRGSDIAG,; | SEQ ID No. 8 |
| LGLNKIVRMYSP,; | SEQ ID No. 9 |
| MGLNKIVRMYSP,; | SEQ ID No. 10 |
| SILDIRQGPKEPFRDYVDRF,; | SEQ ID No. 11 |
| SILDIRQGPKESFRDYVDRF,; | SEQ ID No. 12 |
| SILDIKQGPKEPFRDYVDRF,; | SEQ ID No. 13 |
| SILDIKQGPKESFRDYVDRF,; | SEQ ID No. 14 |
| EEMMTACQGVGGP,; | SEQ ID No. 15 |
| EEMMSACQGVGGP,; | SEQ ID No. 16 |
| PQITLWQRP,; | SEQ ID No. 17 |
| EALLDTGADDTV,; | SEQ ID No. 18 |
| MIGGIGGFIKV,; | SEQ ID No. 19 |
| GCTLNFPISP,; | SEQ ID No. 20 |
| LKPGMDGP,; | SEQ ID No. 21 |
| IGPENPYNTP,; | SEQ ID No. 22 |
| WRKLVDFRELNK,; | SEQ ID No. 23 |
| TQDFWEVQLGIPHP,; | SEQ ID No. 24 |
| SVTVLDVGDAYFS,; | SEQ ID No. 25 |
| FRKYTAFTIPS,; | SEQ ID No. 26 |
| RYQYNVLPQGWKGSP,; | SEQ ID No. 27 |
| DDLYVGSDL,; | SEQ ID No. 28 |
| KHQKEPPFLWMGYELHPD,; | SEQ ID No. 29 |
| WTVNDIQKLVGKLNWASQIY,; | SEQ ID No. 30 |
| EAELELAENREIL,; | SEQ ID No. 31 |
| QWTYQIYQE,; | SEQ ID No. 32 |
| KNLKTGKYA,; | SEQ ID No. 33 |
| YWQATWIP,; | SEQ ID No. 34 |
| NTPPLVKLWY,; | SEQ ID No. 35 |
| VNIVTDSQY,; | SEQ ID No. 36 |
| WVPAHKGIGGNELDCTHLEGK,; | SEQ ID No. 37 |
| LDCTHLEGK,; | SEQ ID No. 38 |
| VAVHVASGY,; | SEQ ID No. 39 |
| LKLAGRWPV,; | SEQ ID No. 40 |
| GIPYNPQSQGV,; | SEQ ID No. 41 |
| TAVQMAVFIHNFKR,; | SEQ ID No. 42 |
| WKGPAKLLWKGEGAVV,; | SEQ ID No. 43 |
| WVTVYYGVPVW,; | SEQ ID No. 44 |
| WATHACVPTDP,; | SEQ ID No. 45 |
| STQLLLNGS,; | SEQ ID No. 46 |
| LTVWGIKQLQ,; | SEQ ID No. 47 |
| IVWQVDRMRI,; | SEQ ID No. 48 |
| ALSEGATP,; | SEQ ID No. 49 |
| ALAEGATP,; | SEQ ID No. 50 |
| HKARVLAE,; | SEQ ID No. 51 |
| HKARILAE,; | SEQ ID No. 52 |
| APRKKGCWAMS,; | SEQ ID No. 53 |
| APRKRGCWAMS,; | SEQ ID No. 54 |
| EGHQMKDCKCG,; | SEQ ID No. 55 |
| EGHQMKECKCG,; | SEQ ID No. 56 |
| HNVWATHACVPTDP,; | SEQ ID No. 57 |
| HNIWATHACVPTDP,; | SEQ ID No. 58 |
| VQCTHGIKPVVSTQLLLNGS,; | SEQ ID No. 59 |
| VQCTHGIKPVISTQLLLNGS,; | SEQ ID No. 60 |
| VQCTHGIRPVVSTQLLLNGS,; | SEQ ID No. 61 |
| VQCTHGIRPVISTQLLLNGS,; | SEQ ID No. 62 |
| LTVWGIKQLQAR,; | SEQ ID No. 63 |
| LTVWGIKQLQAR,; | SEQ ID No. 64 |
| RNRRRRWR,; | SEQ ID No. 65 |
| KNRRRRWR,; | SEQ ID No. 66 |
| IVWQVDRMKI,; | SEQ ID No. 67 |
| and | |
| VGSLQYLAL,. | SEQ ID No. 68 |

17. The HIV vaccine polypeptide of claim 16 comprising at least one copy of at least 90% of the conserved-element peptide sequences.

18. The HIV vaccine polypeptide of claim 17 comprising at least one copy of at least 95% of the conserved-element peptide sequences.

19. An HIV vaccine DNA encoding the HIV vaccine polypeptide of claim 17.

20. The HIV vaccine polypeptide of claim 11 including no HIV proteome peptide fragments not listed in claim 16.


SEQUENCE LISTING (71 sequences)

SEQ ID NO: 1 - protein, 14 aa, Human immunodeficiency virus
PRTLNAWVKVIEEK

SEQ ID NO: 2 - protein, 14 aa, Human immunodeficiency virus
PRTLNAWVKVVEEK

SEQ ID NO: 3 - protein, 14 aa, Human immunodeficiency virus
ARTLNAWVKVIEEK

SEQ ID NO: 4 - protein, 14 aa, Human immunodeficiency virus
ARTLNAWVKVVEEK

SEQ ID NO: 5 - protein, 13 aa, Human immunodeficiency virus
MLNTVGGHQAAMA

SEQ ID NO: 6 - protein, 13 aa, Human immunodeficiency virus
MLNIVGGHQAAMA

SEQ ID NO: 7 - protein, 10 aa, Human immunodeficiency virus
REPRGSDIAG

SEQ ID NO: 8 - protein, 10 aa, Human immunodeficiency virus
RDPRGSDIAG

SEQ ID NO: 9 - protein, 12 aa, Human immunodeficiency virus
LGLNKIVRMYSP

SEQ ID NO: 10 - protein, 12 aa, Human immunodeficiency virus
MGLNKIVRMYSP

SEQ ID NO: 11 - protein, 20 aa, Human immunodeficiency virus
SILDIRQGPKEPFRDYVDRF

SEQ ID NO: 12 - protein, 20 aa, Human immunodeficiency virus
SILDIRQGPKESFRDYVDRF

SEQ ID NO: 13 - protein, 20 aa, Human immunodeficiency virus
SILDIKQGPKEPFRDYVDRF

SEQ ID NO: 14 - protein, 20 aa, Human immunodeficiency virus
SILDIKQGPKESFRDYVDRF

SEQ ID NO: 15 - protein, 13 aa, Human immunodeficiency virus
EEMMTACQGVGGP

SEQ ID NO: 16 - protein, 13 aa, Human immunodeficiency virus
EEMMSACQGVGGP

SEQ ID NO: 17 - protein, 9 aa, Human immunodeficiency virus
PQITLWQRP

SEQ ID NO: 18 - protein, 12 aa, Human immunodeficiency virus
EALLDTGADDTV

SEQ ID NO: 19 - protein, 11 aa, Human immunodeficiency virus
MIGGIGGFIKV

SEQ ID NO: 20 - protein, 10 aa, Human immunodeficiency virus
GCTLNFPISP

SEQ ID NO: 21 - protein, 8 aa, Human immunodeficiency virus
LKPGMDGP

SEQ ID NO: 22 - protein, 10 aa, Human immunodeficiency virus
IGPENPYNTP

SEQ ID NO: 23 - protein, 12 aa, Human immunodeficiency virus
WRKLVDFRELNK

SEQ ID NO: 24 - protein, 14 aa, Human immunodeficiency virus
TQDFWEVQLGIPHP

SEQ ID NO: 25 - protein, 13 aa, Human immunodeficiency virus
SVTVLDVGDAYFS

SEQ ID NO: 26 - protein, 11 aa, Human immunodeficiency virus
FRKYTAFTIPS

SEQ ID NO: 27 - protein, 15 aa, Human immunodeficiency virus
RYQYNVLPQGWKGSP

SEQ ID NO: 28 - protein, 9 aa, Human immunodeficiency virus
DDLYVGSDL

SEQ ID NO: 29 - protein, 18 aa, Human immunodeficiency virus
KHQKEPPFLWMGYELHPD

SEQ ID NO: 30 - protein, 20 aa, Human immunodeficiency virus
WTVNDIQKLVGKLNWASQIY

SEQ ID NO: 31 - protein, 13 aa, Human immunodeficiency virus
EAELELAENREIL

SEQ ID NO: 32 - protein, 9 aa, Human immunodeficiency virus
QWTYQIYQE

SEQ ID NO: 33 - protein, 9 aa, Human immunodeficiency virus
KNLKTGKYA

SEQ ID NO: 34 - protein, 8 aa, Human immunodeficiency virus
YWQATWIP

SEQ ID NO: 35 - protein, 10 aa, Human immunodeficiency virus
NTPPLVKLWY

SEQ ID NO: 36 - protein, 9 aa, Human immunodeficiency virus
VNIVTDSQY

SEQ ID NO: 37 - protein, 12 aa, Human immunodeficiency virus
WVPAHKGIGGNE

SEQ ID NO: 38 - protein, 9 aa, Human immunodeficiency virus
LDCTHLEGK

SEQ ID NO: 39 - protein, 9 aa, Human immunodeficiency virus
VAVHVASGY

SEQ ID NO: 40 - protein, 9 aa, Human immunodeficiency virus
LKLAGRWPV

SEQ ID NO: 41 - protein, 11 aa, Human immunodeficiency virus
GIPYNPQSQGV

SEQ ID NO: 42 - protein, 14 aa, Human immunodeficiency virus
TAVQMAVFIHNFKR

SEQ ID NO: 43 - protein, 16 aa, Human immunodeficiency virus
WKGPAKLLWKGEGAVV

SEQ ID NO: 44 - protein, 11 aa, Human immunodeficiency virus
WVTVYYGVPVW

SEQ ID NO: 45 - protein, 11 aa, Human immunodeficiency virus
WATHACVPTDP

SEQ ID NO: 46 - protein, 9 aa, Human immunodeficiency virus
STQLLLNGS

SEQ ID NO: 47 - protein, 10 aa, Human immunodeficiency virus
LTVWGIKQLQ

SEQ ID NO: 48 - protein, 10 aa, Human immunodeficiency virus
IVWQVDRMRI

SEQ ID NO: 49 - protein, 8 aa, Human immunodeficiency virus
ALSEGATP

SEQ ID NO: 50 - protein, 8 aa, Human immunodeficiency virus
ALAEGATP

SEQ ID NO: 51 - protein, 8 aa, Human immunodeficiency virus
HKARVLAE

SEQ ID NO: 52 - protein, 8 aa, Human immunodeficiency virus
HKARILAE

SEQ ID NO: 53 - protein, 11 aa, Human immunodeficiency virus
APRKKGCWAMS

SEQ ID NO: 54 - protein, 11 aa, Human immunodeficiency virus
APRKRGCWAMS

SEQ ID NO: 55 - protein, 11 aa, Human immunodeficiency virus
EGHQMKDCKCG

SEQ ID NO: 56 - protein, 11 aa, Human immunodeficiency virus
EGHQMKECKCG

SEQ ID NO: 57 - protein, 14 aa, Human immunodeficiency virus
HNVWATHACVPTDP

SEQ ID NO: 58 - protein, 14 aa, Human immunodeficiency virus
HNIWATHACVPTDP

SEQ ID NO: 59 - protein, 20 aa, Human immunodeficiency virus
VQCTHGIKPVVSTQLLLNGS

SEQ ID NO: 60 - protein, 20 aa, Human immunodeficiency virus
VQCTHGIKPVISTQLLLNGS

SEQ ID NO: 61 - protein, 20 aa, Human immunodeficiency virus
VQCTHGIRPVVSTQKKKNGS

SEQ ID NO: 62 - protein, 20 aa, Human immunodeficiency virus
VQCTHGIRPVISTQLLLNGS

SEQ ID NO: 63 - protein, 12 aa, Human immunodeficiency virus
LTVWGIKQLQAR

SEQ ID NO: 64 - protein, 12 aa, Human immunodeficiency virus
LTVWGIKQLQAR

SEQ ID NO: 65 - protein, 8 aa, Human immunodeficiency virus
RNRRRRWR

SEQ ID NO: 66 - protein, 8 aa, Human immunodeficiency virus
KNRRRRWR

SEQ ID NO: 67 - protein, 10 aa, Human immunodeficiency virus
IVWQVDRMKI

SEQ ID NO: 68 - protein, 9 aa, Human immunodeficiency virus
VGSLQYLAL

SEQ ID NO: 69 - DNA, 31 bp, Human immunodeficiency virus
actatgacgctttccatcgggctagctctca

SEQ ID NO: 70 - RNA, 21 nt, Human immunodeficiency virus
acuaugacgcuuuccaucggg

SEQ ID NO: 71 - protein, 6 aa, Human immunodeficiency virus
YDAFHR